(19) United States
(12) Patent Application Publication
(10) Pub. No.: US 2024/0306911 A1
(43) Pub. Date: Sep. 19, 2024

(54) MINIMUM MORPHOLOGICAL REGION ANALYSIS

(71) Applicant: ZOLL Medical Corporation, Chelmsford, MA (US)

(72) Inventors: Jeffery T. Mitchell, Mars, PA (US); Michael A. Sipe, Pittsburgh, PA (US)

(21) Appl. No.: 18/600,836

(22) Filed: Mar. 11, 2024

Related U.S. Application Data
(60) Provisional application No. 63/490,364, filed on Mar. 15, 2023.

(51) Int. Cl. A61B 5/00; A61B 5/259; A61B 5/364

(52) U.S. Cl. CPC A61B 5/0006; A61B 5/259; A61B 5/364

(57) ABSTRACT

A cardiac event monitoring system for applying improved ECG morphological analysis for determining abnormal cardiac events is provided. The system includes a wearable cardiac sensing device including physiological sensors such as ECG electrodes, an ECG digitizing circuit configured to provide digitized ECG signals, and a non-transitory memory configured to store the digitized ECG signals. The system also includes a processor in communication with the wearable cardiac sensing device. The processor is configured to extract a predetermined timeseries of ECG data points from the digitized ECG signals, identify ECG feature data points corresponding to a certain ECG feature, determine a minimum ECG morphological region based on the ECG feature data points, identify one or more measurements corresponding to the certain ECG feature using the minimum ECG morphological region, and determine whether the ECG feature data points correspond to an abnormal cardiac event, based on the one or more measurements.

### CROSS-REFERENCE TO RELATED APPLICATION

This nonprovisional application claims priority to U.S. Provisional Patent Application Ser. No. 63/490,364, filed on Mar. 15, 2023, titled “MINIMUM MORPHOLOGICAL REGION ANALYSIS,” the entirety of which is hereby incorporated by reference.

### BACKGROUND

The present disclosure relates to a wearable cardiac sensing system configured to perform a morphological analysis to determine abnormal cardiac events in a patient.

There are a wide variety of electronic and mechanical devices for monitoring and treating patients' medical conditions. In some examples, depending on the underlying medical condition being monitored or treated, medical devices such as cardiac monitors or defibrillators may be surgically implanted or externally connected to the patient. In some cases, physicians may use medical devices alone or in combination with drug therapies to treat conditions such as cardiac arrhythmias. A patient with a known cardiac condition may be provided with a device that monitors the patient's cardiac activity. The device or a remote server in communication with the device may analyze the patient's cardiac activity as part of monitoring the patient's ongoing cardiac health.

### SUMMARY

In one or more examples, a cardiac event monitoring system for applying improved electrocardiogram (ECG) morphological analysis for determining abnormal cardiac events in a patient is provided. The system includes a wearable cardiac sensing device configured to be bodily-attached to the patient, the wearable cardiac sensing device including a plurality of physiological sensors including one or more ECG electrodes configured to sense electrical signals from a skin surface of the patient, an ECG digitizing circuit configured to provide digitized ECG signals of the patient based on the sensed electrical signals, and a non-transitory memory configured to store the digitized ECG signals of the patient. The system also includes a processor in communication with the wearable cardiac sensing device. The processor is configured to extract a predetermined timeseries of ECG data points from the digitized ECG signals, identify ECG feature data points corresponding to a certain ECG feature from within the digitized ECG signals of the patient, determine a minimum ECG morphological region based on the ECG feature data points, identify one or more measurements corresponding to the certain ECG feature using the minimum ECG morphological region, and determine whether the ECG feature data points correspond to an abnormal cardiac event, based on the one or more measurements corresponding to the certain ECG feature.

Implementations of the cardiac event monitoring system can include one or more of the following features. The minimum ECG morphological region includes a minimum convex ECG morphological region. The minimum convex ECG morphological region includes a plurality of convex vertices and a plurality of line segments interconnecting the plurality of convex vertices and forming an enclosed region. Each of the ECG feature data points is disposed at either a vertex or within the enclosed region. The minimum ECG morphological region includes a plurality of vertices and a plurality of line segments interconnecting the plurality of vertices and forming an enclosed region. At least one of the plurality of vertices is outward-facing. Each of the ECG feature data points is disposed at either a vertex or within the enclosed region. Each of the plurality of vertices is outward facing. The minimum ECG morphological region includes an ellipse configured such that each of the ECG feature data points is disposed at either a boundary of the ellipse or enclosed within the ellipse. The processor is configured to determine the minimum ECG morphological region through morphological closing.

The wearable cardiac sensing device further includes a garment configured to be worn around the patient's torso for an extended period of time. At least a portion of the plurality of physiological sensors are configured to be removably mounted onto the garment. At least a portion of the plurality of physiological sensors are permanently integrated into the garment. The wearable cardiac sensing device further includes a removable adhesive patch configured to be adhered to skin of the patient. At least a portion of the plurality of physiological sensors are configured to be removably mounted onto the removable adhesive patch. The wearable cardiac sensing device further includes a cardiac sensing unit incorporating the portion of the plurality of physiological sensors, the cardiac sensing unit configured to be removably mounted onto the removable adhesive patch.

The wearable cardiac sensing device includes the processor. The wearable cardiac sensing device is in communication with a remote server. The processor is further configured to transmit at least one of the ECG feature data points or an indication of the abnormal cardiac event to the remote server. The wearable cardiac sensing device includes a remote server in communication with the wearable cardiac sensing device. The remote server includes the processor. The wearable cardiac sensing device further includes a portable gateway configured to facilitate communication between the remote server and the wearable cardiac sensing device.

The certain ECG feature includes an RS segment of a QRS complex candidate. The abnormal cardiac event includes a premature ventricular contraction (PVC). The one or more measurements include an area of the minimum ECG morphological region. The ECG feature data points include first ECG feature data points and the minimum ECG morphological region includes a first minimum ECG morphological region based on the first ECG feature data points. The processor is further configured to identify second ECG feature data points corresponding to a QR segment of the QRS complex candidate from within the digitized ECG signals of the patient. The processor is further configured to determine a second minimum ECG morphological region based on the second ECG feature data points and identify one or more measurements corresponding to the QR segment of the QRS complex candidate using the second minimum ECG morphological region. The processor is configured to determine whether the QRS complex candidate corresponds to an abnormal cardiac event, based on the one or more measurements corresponding to the RS segment of the QRS complex candidate and further based on the one or more measurements corresponding to the QR segment of the QRS complex candidate. The one or more measurements corresponding to the RS segment of the QRS complex candidate include an area of the first minimum ECG morphological region, and wherein the one or more measurements corresponding to the QR segment of the QRS complex candidate include an area of the second minimum ECG morphological region. The processor is configured to determine whether the QRS complex candidate corresponds to an abnormal cardiac event, based on a ratio of the area of the first minimum ECG morphological region and the area of the second minimum ECG morphological region.

The certain ECG feature includes a QRS complex. The processor is configured to determine whether the ECG feature data points correspond to an abnormal cardiac event by determining whether the QRS complex includes a notch, based on the one or more measurements corresponding to the QRS complex. The processor is further configured to generate a trace of the ECG feature data points and identify one or more measurements corresponding to the trace of the ECG feature data points. The trace includes a polygon constructed by connecting succeeding ECG feature data points and an enclosing line segment connecting a first of the ECG feature data points and a last of the ECG feature data points. The processor is configured to determine whether the ECG feature data points correspond to an abnormal cardiac event, based on the one or more measurements corresponding to the certain ECG feature and further based on the one or more measurements corresponding to the trace of the ECG feature data points. The one or more measurements corresponding to the QRS complex include an area of the minimum ECG morphological region, and wherein the one or more measurements corresponding to the trace of the ECG feature data points include an area of the trace of the ECG feature data points. The processor is configured to determine whether the ECG feature data points correspond to an abnormal cardiac event, based on a ratio of the trace of the ECG feature data points and the area of the minimum ECG morphological region.

The one or more measurements include an area of the minimum ECG morphological region. The abnormal cardiac event includes a premature ventricular contraction (PVC). The abnormal cardiac event includes a notched QRS complex.

In one or more examples, a cardiac event monitoring system for applying improved electrocardiogram (ECG) morphological analysis for identifying premature ventricular contractions in a patient is provided. The system includes a wearable cardiac sensing device configured to be bodily-attached to the patient, the wearable cardiac sensing device including a plurality of physiological sensors including one or more ECG electrodes configured to sense electrical signals from a skin surface of the patient, an ECG digitizing circuit configured to provide digitized ECG signals of the patient based on the sensed electrical signals, and a non-transitory memory configured to store the digitized ECG signals of the patient. The system also includes a processor in communication with the wearable cardiac sensing device. The processor is configured to extract a predetermined timeseries of ECG data points from the digitized ECG signals, identify ECG feature data points corresponding to an RS segment of a QRS complex candidate from within the digitized ECG signals of the patient, determine a minimum convex ECG morphological region based on the ECG feature data points, identify one or more measurements corresponding to the RS segment of the QRS complex candidate using the minimum convex ECG morphological region, and determine whether the QRS complex candidate corresponds to a premature ventricular contraction (PVC), based on the one or more measurements corresponding to the RS segment of the QRS complex candidate.

Implementations of the wearable cardiac sensing device can include or more of the following features. The one or more measurements include an area of the minimum convex ECG morphological region. The ECG feature data points include first ECG feature data points and the minimum convex ECG morphological region includes a first minimum convex ECG morphological region based on the first ECG feature data points. The processor is further configured to identify second ECG feature data points corresponding to a QR segment of the QRS complex candidate from within the digitized ECG signals of the patient. The processor is further configured to determine a second minimum convex ECG morphological region based on the second ECG feature data points and identify one or more measurements corresponding to the QR segment of the QRS complex candidate using the second minimum convex ECG morphological region. The processor is configured to determine whether the QRS complex candidate corresponds to a PVC, based on the one or more measurements corresponding to the RS segment of the QRS complex candidate and further based on the one or more measurements corresponding to the QR segment of the QRS complex candidate. The one or more measurements corresponding to the RS segment of the QRS complex candidate include an area of the first minimum convex ECG morphological region, and wherein the one or more measurements corresponding to the QR segment of the QRS complex candidate include an area of the second minimum convex ECG morphological region. The processor is configured to determine whether the QRS complex candidate corresponds to a PVC, based on a ratio of the area of the first minimum convex ECG morphological region and the area of the second minimum convex ECG morphological region.

The minimum convex ECG morphological region includes a plurality of convex vertices and a plurality of line segments interconnecting the plurality of convex vertices and forming an enclosed region. Each of the ECG feature data points is disposed at either a vertex or within the enclosed region. The minimum convex ECG morphological region includes an ellipse configured such that each of the ECG feature data points is disposed at either a boundary of the ellipse or enclosed within the ellipse. The processor is configured to determine the minimum convex ECG morphological region through morphological closing.

The wearable cardiac sensing device further includes a garment configured to be worn around the patient's torso for an extended period of time. At least a portion of the plurality of physiological sensors are configured to be removably mounted onto the garment. At least a portion of the plurality of physiological sensors are permanently integrated into the garment. The wearable cardiac sensing device further includes a removable adhesive patch configured to be adhered to skin of the patient. At least a portion of the plurality of physiological sensors are configured to be removably mounted onto the removable adhesive patch. The wearable cardiac sensing device further includes a cardiac sensing unit incorporating the portion of the plurality of physiological sensors, the cardiac sensing unit configured to be removably mounted onto the removable adhesive patch.

The wearable cardiac sensing device includes the processor. The wearable cardiac sensing device is in communication with a remote server. The processor is further configured to transmit at least one of the ECG feature data points or an indication of the abnormal cardiac event to the remote server. The system includes a remote server in communication with the wearable cardiac sensing device. The remote server includes the processor. The wearable cardiac sensing device further includes a portable gateway configured to facilitate communication between the remote server and the wearable cardiac sensing device.

In one or more examples, a cardiac event monitoring system for applying improved electrocardiogram (ECG) morphological analysis for identifying notched QRS complexes in a patient is provided. The system includes a wearable cardiac sensing device configured to be bodily-attached to the patient, the wearable cardiac sensing device including a plurality of physiological sensors including one or more ECG electrodes configured to sense electrical signals from a skin surface of the patient, an ECG digitizing circuit configured to provide digitized ECG signals of the patient based on the sensed electrical signals, and a non-transitory memory configured to store the digitized ECG signals of the patient. The system includes a processor in communication with the wearable cardiac sensing device. The processor is configured to extract a predetermined timeseries of ECG data points from the digitized ECG signals, identify ECG feature data points corresponding to a QRS complex from within the digitized ECG signals of the patient, generate a trace of the ECG feature data points, identify one or more measurements corresponding to the trace of the ECG feature data points, determine a minimum convex ECG morphological region based on the ECG feature data points, identify one or more measurements corresponding to the QRS complex using the minimum convex ECG morphological region, and determine whether the QRS complex includes a notch, based on the one or more measurements corresponding to the trace of the ECG feature data points and on the one or more measurements corresponding to the QRS complex.

Implementations of the wearable cardiac monitoring device can include one or more of the following features. The trace includes a polygon constructed by connecting succeeding ECG feature data points and an enclosing line segment connecting a first of the ECG feature data points and a last of the ECG feature data points. The one or more measurements corresponding to the QRS complex include an area of the minimum convex ECG morphological region. The one or more measurements corresponding to the trace of the ECG feature data points include an area of the trace of the ECG feature data points. The processor is configured to determine whether the QRS complex includes a notch, based on a ratio of the area of the trace of the ECG feature data points and the area of the minimum convex ECG morphological region.

The minimum convex ECG morphological region includes a plurality of convex vertices and a plurality of line segments interconnecting the plurality of convex vertices and forming an enclosed region. Each of the ECG feature data points is disposed at either a vertex or within the enclosed region. The minimum convex ECG morphological region includes an ellipse configured such that each of the ECG feature data points is disposed at either a boundary of the ellipse or enclosed within the ellipse. The processor is configured to determine the minimum convex ECG morphological region through morphological closing.

The wearable cardiac sensing device further includes a garment configured to be worn around the patient's torso for an extended period of time. At least a portion of the plurality of physiological sensors are configured to be removably mounted onto the garment. At least a portion of the plurality of physiological sensors are permanently integrated into the garment. The wearable cardiac sensing device further includes a removable adhesive patch configured to be adhered to skin of the patient. At least a portion of the plurality of physiological sensors are configured to be removably mounted onto the removable adhesive patch. The wearable cardiac sensing device further includes a cardiac sensing unit incorporating the portion of the plurality of physiological sensors, the cardiac sensing unit configured to be removably mounted onto the removable adhesive patch.

The wearable cardiac sensing device includes the processor. The wearable cardiac sensing device is in communication with a remote server. The processor is further configured to transmit at least one of the ECG feature data points or an indication of the abnormal cardiac event to the remote server. The system further includes a remote server in communication with the wearable cardiac sensing device. The remote server includes the processor. The wearable cardiac sensing device further includes a portable gateway configured to facilitate communication between the remote server and the wearable cardiac sensing device.

In one or more examples, a method for applying improved electrocardiogram (ECG) morphological analysis for determining abnormal cardiac events in a patient is implemented. The method includes extracting a predetermined timeseries of ECG data points from digitized ECG signals, identifying ECG feature data points corresponding to a certain ECG feature from within the digitized ECG signals of the patient, determining a minimum ECG morphological region based on the ECG feature data points, identifying one or more measurements corresponding to the certain ECG feature using the minimum ECG morphological region, and determining whether the ECG feature data points correspond to an abnormal cardiac event, based on the one or more measurements corresponding to the certain ECG feature.

In one or more examples, a non-transitory computer-readable medium storing sequences of instructions executable by at least one processor is provided. The sequences of instructions instruct the at least one processor to extract a predetermined timeseries of ECG data points from digitized ECG signals, identify ECG feature data points corresponding to a certain ECG feature from within the digitized ECG signals of a patient, determine a minimum ECG morphological region based on the ECG feature data points, identify one or more measurements corresponding to the certain ECG feature using the minimum ECG morphological region, and determine whether the ECG feature data points correspond to an abnormal cardiac event, based on the one or more measurements corresponding to the certain ECG feature.

In one or more examples, a method for applying improved electrocardiogram (ECG) morphological analysis for identifying premature ventricular contractions in a patient is implemented. The method includes extracting a predetermined timeseries of ECG data points from digitized ECG signals, identifying ECG feature data points corresponding to an RS segment of a QRS complex candidate from within the digitized ECG signals of the patient, determining a minimum convex ECG morphological region based on the ECG feature data points, identifying one or more measurements corresponding to the RS segment of the QRS complex candidate using the minimum convex ECG morphological region, and determining whether the QRS complex candidate corresponds to a premature ventricular contraction (PVC), based on the one or more measurements corresponding to the RS segment of the QRS complex candidate.

In one or more examples, a non-transitory computer-readable medium storing sequences of instructions executable by at least one processor is provided. The sequences of instructions instruct the at least one processor to extract a predetermined timeseries of ECG data points from digitized ECG signals, identify ECG feature data points corresponding to an RS segment of a QRS complex candidate from within the digitized ECG signals of a patient, determine a minimum convex ECG morphological region based on the ECG feature data points, identify one or more measurements corresponding to the RS segment of the QRS complex candidate using the minimum convex ECG morphological region, and determine whether the QRS complex candidate corresponds to a premature ventricular contraction (PVC), based on the one or more measurements corresponding to the RS segment of the QRS complex candidate.

In one or more examples, a method for applying improved electrocardiogram (ECG) morphological analysis for identifying notched QRS complexes in a patient is implemented. The method includes extracting a predetermined timeseries of ECG data points from digitized ECG signals, identifying ECG feature data points corresponding to a QRS complex from within the digitized ECG signals of the patient, generating a trace of the ECG feature data points, identifying one or more measurements corresponding to the trace of the ECG feature data points, determining a minimum convex ECG morphological region based on the ECG feature data points, identifying one or more measurements corresponding to the QRS complex using the minimum convex ECG morphological region, and determining whether the QRS complex includes a notch, based on the one or more measurements corresponding to the trace of the ECG feature data points and on the one or more measurements corresponding to the QRS complex.

In one or more examples, a non-transitory computer-readable medium storing sequences of instructions executable by at least one processor is provided. The sequences of instructions instruct the at least one processor to extract a predetermined timeseries of ECG data points from digitized ECG signals, identify ECG feature data points corresponding to a QRS complex from within the digitized ECG signals of a patient, generate a trace of the ECG feature data points, identify one or more measurements corresponding to the trace of the ECG feature data points, determine a minimum convex ECG morphological region based on the ECG feature data points, identify one or more measurements corresponding to the QRS complex using the minimum convex ECG morphological region, and determine whether the QRS complex includes a notch, based on the one or more measurements corresponding to the trace of the ECG feature data points and on the one or more measurements corresponding to the QRS complex.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiac sensing system including a wearable cardiac sensing device.

FIG. 2 depicts an example adhesive patch of a wearable cardiac sensing device.

FIG. 3 depicts an example cardiac sensing unit of a wearable cardiac sensing device.

FIG. 4 depicts an example of using an adhesive patch with a cardiac sensing unit.

FIG. 5 depicts an example electronic architecture for a cardiac sensing unit.

FIG. 6 depicts another example wearable cardiac sensing system including a wearable cardiac sensing device.

FIG. 7 depicts an example garment-based monitoring device of a wearable cardiac sensing device.

FIG. 8 depicts an example electronic architecture for a garment-based monitoring device.

FIG. 9 depicts an example process flow for applying an improved electrocardiogram (ECG) morphological analysis for determining abnormal cardiac events.

FIG. 10A depicts a sample set of data points.

FIG. 10B depicts determining a minimum morphological region for the sample set of data points of FIG. 10A.

FIG. 10C depicts determining a minimum morphological region for the sample set of data points of FIG. 10A.

FIG. 10D depicts determining a minimum morphological region for the sample set of data points of FIG. 10A.

FIG. 10E depicts determining a minimum morphological region for the sample set of data points of FIG. 10A.

FIG. 10F depicts determining a minimum morphological region for the sample set of data points of FIG. 10A.

FIG. 11A depicts a sample set of ECG data points.

FIG. 11B depicts a minimum ECG morphological region for the sample set of ECG data points of FIG. 11A.

FIG. 11C depicts another minimum ECG morphological region for the sample set of ECG data points of FIG. 11A.

FIG. 12 depicts an example process flow for applying an improved ECG morphological analysis for identifying premature ventricular contractions (PVCs).

FIG. 13 depicts an example of minimum ECG morphological regions in a QRS complex candidate.

FIG. 14 depicts example histogram constructed for normal and PVC beats.

FIG. 15 depicts an example plot constructed for normal and PVC beats.

FIG. 16 depicts an example process flow for applying an improved ECG morphological analysis for identifying notched QRS complexes.

FIG. 17 depicts an example of a minimum ECG morphological region and a trace in a QRS complex.

FIG. 18A depicts an example screen that may be displayed to a patient.

FIG. 18B depicts another example screen that may be displayed to a patient.

FIG. 18C depicts another example screen that may be displayed to a patient.

FIG. 19 depicts an example component view of a cardiac sensing unit.

FIG. 20 depicts another example of a wearable cardiac sensing device.

FIG. 21 depicts another example of a wearable cardiac sensing device.

FIG. 22 depicts another example of a wearable cardiac sensing device.

### DETAILED DESCRIPTION

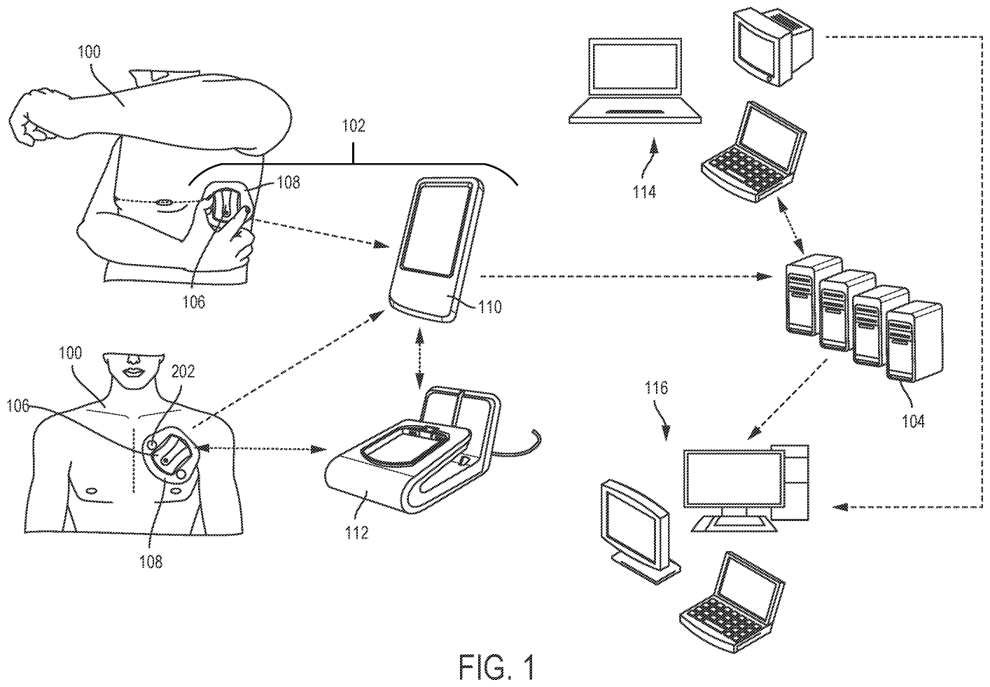

Cardiac event monitoring systems implementing the devices, methods, and techniques disclosed herein can be used to monitor patients with known or suspected cardiac conditions. For example, some patients with a suspected cardiac condition may be prescribed a wearable cardiac monitor, or a wearable cardiac monitor and treatment device, that the patient must wear for the prescribed period of time. During the period of wear, the cardiac monitor generates ECG signals for the patient and may collect other data about the patient, such as data regarding the patient's movement, posture, lung fluid levels, respiration rate, and/or the like. The ECG signals are analyzed, at the cardiac monitor and/or at a remote server in communication with the cardiac monitor, to determine if the patient has a cardiac condition. Example cardiac conditions may include arrhythmias such as ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2^{nd} AV blocks, 3^{rd} AV blocks, bradycardia, and/or other types of tachycardia. To illustrate, referring to FIG. 1, a remote server 104 in remote communication with a wearable cardiac sensing device 102 may perform other processing or analyses of the ECG signal received from the wearable cardiac sensing device 102, such as band pass filtering, detecting R-R intervals, detecting QRS intervals, and/or heart rate estimation. Additionally or alternatively, a patient with a known cardiac condition may be prescribed a wearable cardiac monitor and treatment device configured to provide the patient with a treatment, such as therapeutic defibrillation pulses, upon detecting certain types of treatable arrhythmias (e.g., ventricular fibrillation). The patient may similarly use the wearable cardiac monitor and treatment device for the prescribed period of time, while the device and/or remote server analyzes the patient's ECG signals to determine cardiac conditions in the patient.

As discussed in further detail below, the cardiac event monitoring systems implementing the devices, methods, and techniques disclosed herein may provide improved ECG signal analysis to determine abnormal cardiac conditions in a patient. More specifically, the cardiac event monitoring systems implementing the devices, methods, and techniques disclosed herein may apply ECG morphological analyses to determine abnormal cardiac events in the patient. The ECG morphological analyses described herein may include determining a minimum ECG morphological region for certain features of the patient's ECG, such as the patient's QRS complex, QR segment, RS segment, and/or the like. Using the minimum ECG morphological region, the cardiac event monitoring system may be able to identify, or use as an input in a beat or cardiac event classifier to identify, certain abnormal cardiac events in the patient.

In implementations, the cardiac event monitoring system includes a wearable cardiac sensing device configured to be bodily-attached to the patient. Examples of wearable cardiac sensing devices may include an adhesive device configured to be adhered to the skin of the patient or a garment-based device configured to be worn under the patient's clothing for an extended period of time. The wearable cardiac sensing device includes a number of physiological sensors, including one or more ECG electrodes configured to sense electrical signals from a skin surface of the patient. The wearable cardiac sensing device also includes an ECG digitizing circuit configured to provide digitized signals of the patient based on the sensed electrical signals that are stored in a non-transitory memory of the wearable cardiac sensing device. Other physiological sensors of the wearable cardiac sensing device may include, for instance, a motion sensor such as an accelerometer, a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The cardiac event monitoring system also includes a processor in communication with the wearable cardiac sensing device. The processor may be located at the wearable cardiac sensing device or at a remote server in communication with the wearable cardiac sensing device. Alternatively, the functions of the processor may be split between the wearable cardiac sensing device and the remote server. The processor is configured to extract a predetermined timeseries of ECG data points from the digitized ECG signals and identify ECG feature data points corresponding to a certain ECG feature from within the digitized ECG signals of the patient. For example, the certain ECG feature may be a QRS complex or a feature considered a QRS complex candidate that needs to be confirmed as a normal QRS complex or corresponding to an abnormal cardiac beat, such as a premature ventricular contraction.

The processor is further configured to determine a minimum ECG morphological region based on the ECG feature data points. For example, the minimum ECG morphological region may be a region constructed with a minimum amount of area that includes all of the ECG feature data points either within the region or on a boundary or boundaries of the region. In examples, the minimum ECG morphological region may be constructed using convex hull methods, though other methods for generating a minimum ECG morphological region may also be used as described below. The processor is configured to identify one or more measurements corresponding to the certain ECG feature using the minimum ECG morphological region and determine whether the ECG feature data points correspond to an abnormal cardiac event, based on the one or more measurements corresponding to the certain ECG feature. As an illustration, the processor may use the area of the minimum ECG morphological region to determine whether the certain ECG feature is a PVC, whether the certain ECG feature contains a notch, and/or the like.

In one example use case, a cardiologist may prescribe that a patient with a suspected cardiac condition use a wearable cardiac sensing device for a prescribed time period. The wearable cardiac sensing device may include an adhesive patch, a cardiac sensing unit configured to be mounted onto the adhesive patch, and a portable gateway in communication with the cardiac sensing unit and with a remote server. The patient may use the wearable cardiac sensing device during their daily activities (e.g., with the adhesive patch and cardiac sensing unit worn under the patient's clothes and the portable gateway carried with the patient) during the prescribed time period. As the wearable cardiac sensing device is being used by the patient, the wearable cardiac sensing device provides ECG signals for the patient based on sensed electrical activity. At the portable gateway and/or at the remote server, the ECG signals may be subjected to a morphological analysis. For example, in such a morphological analysis, a minimum ECG morphological region is created for certain features of the ECG signals as described in further detail below. Based on the minimum ECG morphological region, the portable gateway and/or the remote server may determine if the patient is expressing abnormal cardiac events, such as PVCs or notched or fragmented QRS complexes.

In implementations, the determination as to whether the patient is expressing abnormal cardiac events can be carried out on the gateway device. In implementations, the determination as to whether the patient is expressing abnormal cardiac events can be carried out on the remote server. In implementations, the determination as to whether the patient is expressing abnormal cardiac events can be carried out on both the gateway and the remote server. In implementations, some portion of the determination whether the patient is expressing abnormal cardiac events can be carried on the gateway, and another portion of such determination can be carried out at the remote server. In implementations, an initial determination whether the patient is expressing abnormal cardiac events can be carried on the gateway, and a confirmation (e.g., verification) of the initial determination can be carried out at the remote server. In some or all such implementations, the remote server may then prepare reports for the patient's cardiologist summarizing the patient's abnormal cardiac events. For example, such reports can be displayed on a cardiac dashboard via a web-based portal with access to the remote server, presenting information on a number of patients being overseen by the cardiologist, or the reports may otherwise be transmitted to the cardiologist. For instance, the remote server may send electronic mail (email) reports summarizing the patient's abnormal cardiac events to the patient's cardiologist.

In another example use case, a cardiologist may prescribe a patient with a known cardiac condition a wearable cardiac sensing device with treatment capabilities. For example, the wearable cardiac sensing device may be configured to provide cardioversion, defibrillation, and/or pacing (e.g., anti-bradycardic or anti-tachycardic) treatment pulses to the patient upon detecting a treatable arrhythmia in the patient. The wearable cardiac sensing and treatment device may also include a portable gateway that in turn is in communication with a remote server. Alternatively, the wearable cardiac sensing and treatment device may not include a portable gateway as an intermediary communication device. Instead, the wearable cardiac sensing and treatment device may be in direct communication with the remote server (e.g., be configured as a garment-based monitoring and treatment device in direct communication with the remote server). In implementations, the wearable cardiac sensing device may be configured as a garment-based sensing device including a garment where monitoring and treatment components of the garment-based sensing device are configured to be removably mounted onto the garment. Alternatively, at least some of the monitoring and treatment components of the garment-based sensing device may be permanently disposed on the garment. For example, the garment may include ECG electrodes and/or treatment electrodes sewn into, adhered onto, incorporated into the threads of, or otherwise permanently included as part of the garment. The patient wears the garment-based sensing device under their clothes for the prescription period, which may be the period between when the patient is diagnosed with the known cardiac condition and when the patient is scheduled to receive an implantable treatment device for the cardiac condition, such as an implantable defibrillator. In other situations, the patient may wear the garment-based sensing device for a period of time as the patient undergoes cardiac rehabilitation to improve their cardiac condition to the point that they no longer need to be protected by a treatment device. Alternatively, in some implementations, the wearable cardiac sensing device may be configured as an adhesive patch removably attached to the body of the patient and configured to support both monitoring and treatment components disposed on the patch.

In implementations, the determination as to whether the patient is expressing abnormal cardiac events can be carried out on the portable gateway device. In implementations, the determination as to whether the patient is expressing abnormal cardiac events can be carried out on the remote server. In implementations, the determination as to whether the patient is expressing abnormal cardiac events can be carried out on both the gateway and the remote server. In implementations, some portion of the determination as to whether the patient is expressing abnormal cardiac events can be carried on the gateway, and another portion of such determination can be carried out at the remote server. In implementations, an initial determination as to whether the patient is expressing abnormal cardiac events can be carried on the gateway, and a confirmation (e.g., verification) of the initial determined can be carried out at the remote server. If the arrhythmia is determined to be life-threatening, the device can issue one or more appropriate treatment pulses to the patient.

In implementations, the determination as to whether the patient is expressing abnormal cardiac events can be carried out on the cardiac sensing and treatment device (e.g., without communicating or with minimal communication with the remote server). As part of monitoring the patient for potentially life-threatening cardiac events, the wearable cardiac sensing device may also perform morphological analysis on the ECG signals generated by the device to detect the abnormal life-threatening cardiac events. The wearable cardiac sensing device may also send indications of detected abnormal cardiac events to a remote server in communication with the wearable cardiac sensing device, which may alert the patient's cardiologist of the detected abnormal events (e.g., through a cardiac dashboard, through an email or text message, etc.).

The cardiac event monitoring systems described herein may provide advantages over prior art systems. For example, determining and analyzing a minimum ECG morphological region may provide outputs that, in turn, can help cardiac event classifiers more accurately identify abnormal cardiac beats or other events, as described in further detail below. Furthermore, an identified minimum ECG morphological region may be more sensitive to certain morphological features of an ECG signal than using, for instance, a trace of the ECG data points, as the trace may be more sensitive and thus susceptible to the presence of high-frequency or short-lived noise artifacts. By more accurately identifying abnormal cardiac beats, the cardiac event monitoring systems may help cardiologists with correctly diagnosing cardiac issues in patients, which may help patients experience better long-term health outcomes. Additionally, cardiac event monitoring systems that also provide therapeutic treatments to patient may show improvements in correctly detecting treatable, life-threatening cardiac arrhythmias as opposed to non-treatable cardiac arrhythmias or non-life threatening cardiac arrhythmias. As such, patients may be better protected by these systems and also be less at a risk of experiencing false positive detections, which may require the patient to intervene (e.g., by pressing a response button) to avoid being shocked.

FIG. 1 illustrates an example of a cardiac event monitoring system for applying a morphological region analysis on signals gathered from a patient 100, according to implementations disclosed herein. The cardiac event monitoring system of FIG. 1 includes a wearable cardiac sensing device 102 configured for to be bodily-attached to the patient 100 (e.g., for long-term continuous wear by the patient 100), where the wearable cardiac sensing device 102 is in communication with a remote server 104. The wearable cardiac sensing device 102 includes a number of physiological sensors. As an example, the physiological sensors may include one or more ECG electrodes configured to sense electrical signals from a skin surface of the patient 100. In the example of FIG. 1, the wearable cardiac sensing device 102 includes a cardiac sensing unit 106 removably coupled to a removable adhesive patch 108. However, other implementations of the wearable cardiac sensing system may include different embodiments of a wearable cardiac sensing device 102, as described in further detail below.

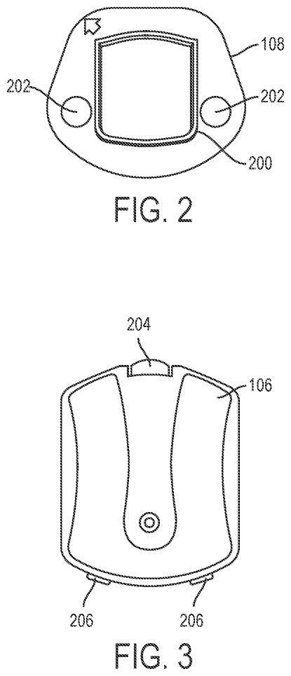

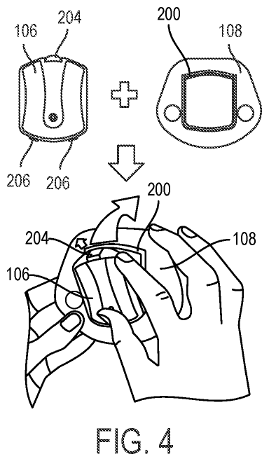

The adhesive patch 108 is configured to be adhesively attached or coupled to the skin of the patient 100. The adhesive patch 108 is further configured such that the cardiac sensing unit 106 may be removably attached to the adhesive patch 108. For example, referring to FIG. 2, the adhesive patch 108 may include a frame 200 (e.g., a plastic frame) delineating the boundary of the region of the adhesive patch 108 that is configured for receiving the cardiac sensing unit 106. The adhesive patch 108 may be disposable (e.g., single-, few-, or multiple-use patches) and may be made of biocompatible, non-woven material. In some embodiments, the cardiac sensing unit 106 may be designed for long-term, continuous usage. In such embodiments, the connection between the cardiac sensing unit 106 and the adhesive patch 108 may be configured to be reversible. For example, the cardiac sensing unit 106 may be configured to be removably attached to the adhesive patch 108. In implementations, as shown in FIG. 3, the cardiac sensing unit 106 may include components such as a snap-in clip 204 that is configured to secure the cardiac sensing unit 106 to the adhesive patch 108 upon attachment to the patch frame 200. After the cardiac sensing unit 106 is attached to the frame 200, a user may press the snap-in clip 204 to subsequently release the cardiac sensing unit 106 from the frame 200. In implementations, as further shown in FIG. 3, the cardiac sensing unit 106 may include positioning tabs 206 that facilitate the attachment process between the cardiac sensing unit 106 and the adhesive patch 108. For example, the positioning tabs 206 may guide a user to insert the cardiac sensing unit 106 onto the correct portion of the frame 200 such that the cardiac sensing unit 106 can then be coupled, connected, or snapped into the frame 200 using the snap-in clip 204, as shown in FIG. 4.

Referring back to FIG. 1, FIG. 1 illustrates example locations on the body of the patient 100 where an adhesive patch 108 housing the cardiac sensing unit 106 may be placed. As an example, the adhesive patch 108 may be placed on the side of the patient's thorax (e.g., under the patient's armpit, level with a circumferential line running across the patient's chest). As another example, the adhesive patch 108 may be placed on the front of the patient's thorax (e.g., to the left of a medial line running between the patient's collarbone, for patients without dextrocardia). However, the adhesive patch 108 may also be placed on any part of the patient's thorax that allows for efficient monitoring and recording of signals from and/or associated with the patient 100. For instance, the adhesive patch 108 may be placed over the patient's sternum, over a lower portion of the patient's front or side thorax (e.g., under the patient's sternum), and/or the like.

The cardiac sensing unit 106 and adhesive patch 108 are configured for long-term and/or extended use or wear by, or attachment or connection to, the patient 100. For example, devices as described herein are capable of being continuously used or continuously worn by, or attached or connected to, the patient 100 without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient 100 is resumed. As an illustration, the cardiac sensing unit 106 may be removed for charging, to carry out technical service, to update the device software or firmware, for the patient 100 to take a shower, and/or for other reasons or activities without departing from the scope of the examples described herein. As another illustration, the patient 100 may remove a used adhesive patch 108, as well as the cardiac sensing unit 106, so that the patient 100 may adhere a new adhesive patch 108 to their body and attach the cardiac sensing unit 106 to a new adhesive patch 108. Such substantially or nearly continuous use, monitoring, or wear as described herein may nonetheless be considered continuous use, monitoring, or wear.

For example, in implementations, the adhesive patch 108 may be designed to maintain attachment to skin of the patient 100 for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 108 can be removed from the patient's skin and the cardiac sensing unit 106 can be removed from the adhesive patch 108. The cardiac sensing unit 106 can then be removably coupled, connected, or snapped onto a new adhesive patch 108 and reapplied to the patient's skin.

As further shown in FIG. 1, the wearable cardiac sensing device 102 may include a portable gateway 110 configured to facilitate communication between the wearable cardiac sensing device 102 and the remote server 104. In implementations, the portable gateway 110 is configured to receive data and/or signals provided by the wearable cardiac sensing device 102 and transmit the data and/or signals to the remote server 104. As an illustration, in implementations, the portable gateway 110 may communicate with the remote server 104 via Ethernet, via Wi-Fi, via near-field communication (NFC), via radiofrequency, via cellular networks, via Bluetooth®-to-TCP/IP access point communication, and/or the like. Accordingly, the portable gateway 110 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, the wearable cardiac sensing device 102 and/or the portable gateway 110 may include communications circuitry that is part of an Internet of Things (IoT) and communicate with each other and/or the remote server 104 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in other implementations, the wearable cardiac sensing device 102 may be configured to transmit data and/or signals directly to the remote server 104 instead of, or in addition to, transmitting the signals to the portable gateway 110. Accordingly, the wearable cardiac sensing device 102 may be in wired or wireless communication with the remote server 104. As an illustration, the wearable cardiac sensing device 102 may communicate with the remote server 104 via Ethernet, via Wi-Fi, via near-field communication (NFC), via radiofrequency, via cellular networks, via Bluetooth®-to-TCP/IP access point communication, and/or the like. Further, in some implementations, the cardiac event monitoring system may not include the portable gateway 110. In such implementations, the wearable cardiac sensing device 102 may perform the functions of the portable gateway 110 described herein. Additionally, in implementations where the wearable cardiac sensing device 102 is configured to communicate directly with the remote server 104, the wearable cardiac sensing device 102 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, as indicated above, the communications circuitry in the wearable cardiac sensing device 102 may be part of an IoT and communicate with the remote server 104 via IoT protocols for handling secure (e.g., encrypted) messaging and routing.

The wearable cardiac sensing system may also include a charger 112, as further shown in FIG. 1. In implementations, the charger 112 includes charging cradles configured to hold and recharge the cardiac sensing unit 106 and the portable gateway 110. Alternatively, in some implementations, the cardiac event monitoring system may not include the portable gateway 110, and accordingly, the charger 112 may be configured to hold the cardiac sensing unit 106 alone. In implementations, the cardiac sensing unit 106 and the portable gateway 110 may have separate chargers, or one or both of the cardiac sensing unit 106 and the portable gateway 110 may have removable batteries that may be replaced and/or recharged.

The remote server 104 is configured to receive and process the data and/or signals received from the wearable cardiac sensing device 102. In implementations, the remote server 104 may be in electronic communication with a number of wearable cardiac sensing devices 102 and be configured to receive and process the data and/or signals received from all of the wearable cardiac sensing devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiac sensing device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others. In various implementations, the remote server 104 may use the data received from the wearable cardiac sensing device 102 to determine a current status of cardiotoxicity in the patient 100, as described in further detail below. Alternatively, in some implementations, the wearable cardiac sensing device 102 may perform some or all of the analysis described herein as being performed by the remote server 104 (e.g., with the wearable cardiac sensing device 102 transmitting an indication of an abnormal cardiac event in the patient 100 or another output to the remote server 104, for instance, via the portable gateway 110).

The remote server 104 is configured to receive and process the data and/or signals received from the wearable cardiac sensing device 102. In implementations, the remote server 104 may be in electronic communication with a number of wearable cardiac sensing devices 102 and be configured to receive and process the data and/or signals received from all of the wearable cardiac sensing devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiac sensing device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others.

As shown in FIG. 1, in implementations, the cardiac event monitoring system further includes one or more user interfaces, such as technician interfaces 114 and caregiver interfaces 116. The technician interfaces 114 and caregiver interfaces 116 are in electronic communication with the remote server 104 through a wired or wireless connection. For instance, the technician interfaces 114 and caregiver interfaces 116 may communicate with the remote server 104 via Wi-Fi, via Ethernet, via cellular networks, and/or the like. Additionally, as shown, at least some of the technician interfaces 114 may be in electronic communication with at least some of the caregiver interfaces 116 through a wired or wireless connection, such as via Wi-Fi, via Ethernet, via cellular networks, and/or the like. The technician interfaces 114 and the caregiver interfaces 116 may include, for example, desktop computers, laptop computers, and/or portable personal digital assistants (e.g., smartphones, tablet computers, etc.).

In implementations, the technician interfaces 114 are configured to electronically communicate with the remote server 104 for the purpose of viewing and analyzing information gathered from one or more wearable cardiac sensing devices 102. For example, a technician interface 114 may provide one or more instructions to the remote server 104 to prepare a report on data and/or signals received from a given wearable cardiac sensing device 102 for a certain time period. Accordingly, a technician interface 114 may include a computing device having a processor communicably connected to a memory and a visual display. The technician interface 114 may display to a user of the technician interface 114 (e.g., a technician) data received from a wearable cardiac sensing device 102 and/or information computed from the data and/or signals received from wearable cardiac sensing device 102, described in further detail below. The user of the technician interface 114 may then provide one or more inputs to the remote server 104 to guide the remote server 104 in, for example, preparing a report for a patient 100.

As an illustration, a user of a technician interface 114 may select a time period to use for a report, and the remote server 104 may prepare a report corresponding to the selected time period. As another example, a user of a technician interface 114 may select types of data to be included in a report, such as certain abnormal cardiac events as described in more detail below. The remote server 104 may then prepare a report according to the types of data selected by the user. As another example, a user of a technician interface 114 may view a report prepared by the remote server 104 and draft a summary of the report to be included in a summary section for the report. Alternatively, in implementations, the remote server 104 may analyze, summarize, etc. the data and/or signals received from a wearable cardiac sensing device 102 with minimal or no input or interaction with a technician interface 114. In this way, the remote server 104 may analyze, summarize, etc. the information gathered from a wearable cardiac sensing device 102 and prepare a report on this information through a completely or mostly automated process.

The caregiver interfaces 116 are configured to electronically communicate with the remote server 104 for the purpose of viewing information on various patients 100 using a wearable cardiac sensing device 102. As such, a caregiver interface 116 may include a computing device having a processor communicably connected to a memory and a visual display. The caregiver interface 116 may display to a user of the caregiver interface 116 (e.g., a physician, a nurse, or other caregiver), for example, abnormal cardiac events detected for the patient 100 using the wearable cardiac sensing device 102, as described in further detail below. In implementations, the caregiver interface 116 may display to a user one or more reports summarizing abnormal cardiac events in the patient 100, such as one or more reports prepared by the remote server 104 (e.g., based on inputs from one or more technician interfaces 114). In implementations, the user of a caregiver interface 116 may be able to interact with the information displayed on the caregiver interface 116. As an example, the user of a caregiver interface 116 may be able to select a portion of a patient report and, in response, be able to view additional information relating to the selected portion of the report. Additional information may include, for instance, ECG data from the wearable cardiac sensing device 102 used to prepare the report and/or the like. In implementations, the user of the caregiver interface 116 may instead view a static patient report that does not have interactive features.

In implementations, a technician interface 114 and/or a caregiver interface 116 may be a specialized interface configured to communicate with the remote server 104. As an example, the technician interface 114 may be a specialized computing device configured to receive preliminary patient reports from the remote server 104, receive inputs from a user to adjust the preliminary report, and transmit the inputs back to the remote server 104. The remote server 104 then uses the inputs from the technician interface 114 to prepare a finalized patient report, which the remote server 104 also transits to the technician interface 114 for review by the user. As another example, the caregiver interface 116 may be a specialized computing device configured to communicate with the remote server 104 to receive and display patient reports, as well as other information regarding patients 100 using a wearable cardiac sensing device 102.

In implementations, a technician interface 114 and/or a caregiver interface 116 may be a generalized user interface that has been adapted to communicate with the remote server 104. To illustrate, the technician interface 114 may be a computing device (e.g., a laptop, a portable personal digital assistant such as a smartphone or tablet, etc.) executing a technician application that configures the computing device to communicate with the remote server 104. For example, the technician application may be downloaded from an application store or otherwise installed on the computing device. Accordingly, when the computing device executes the technician application, the computing device is configured to establish an electronic communication link with the remote server 104 to receive and transmit information regarding patients 100 using a wearable cardiac sensing device 102. Similarly, the caregiver interface 116 may be a computing device (e.g., a laptop, a portable personal digital assistant such as a smartphone or tablet, etc.) executing a caregiver application that configures the computing device to communicate with the remote server 104. The caregiver application may be similarly downloaded from an application store or otherwise installed on the computing device and, when executed, may configure the computing device to establish a communication link with the remote server 104 to receive and display information on patients 100 using a wearable cardiac sensing device 102.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other cases, the technician application and the caregiver application may be separate applications, each providing separate functionalities to a computing device executing them.

In implementations, the cardiac event monitoring system shown in FIG. 1 may include other types of interfaces. To illustrates, in examples, the cardiac event monitoring system may include patient interfaces. The remote server 104 and/or a technician interface 114 may provide a report on a patient 100 using a wearable cardiac sensing device 102 to this patient 100 via a patient interface. This patient report may be the same as a report provided to a caregiver via a caregiver interface 116, or this report may be different from the report provided to the caregiver via the caregiver interface 116. For instance, the report provided to a patient 100 may be an abridged version of a report prepared for the patient's caregiver, such as a report that summarizes the key information from the report prepared for the patient's caregiver. In various implementations, the patient interface may be configured similarly to and function similarly to the caregiver interface 116 discussed above. As an example, the patient interface may be similar to the caregiver interface 116 but with some restrictions on what is included in a patient report compared to a report prepared for the patient's caregiver, and/or with some restrictions on the functionalities of, for instance, an application executed by the patient interface that the patient 100 can use to access and view a patient report or other information on the patient 100.

Returning back to the wearable cardiac sensing device 102, as noted above the wearable cardiac sensing device 102 includes a number of physiological sensors configured to sense signals from and/or associated with the patient 100. To illustrate, in embodiments of the wearable cardiac sensing device 102 that includes a cardiac sensing unit 106 and an adhesive patch 108, as described above with reference to FIG. 1, the physiological sensors may be implemented in the cardiac sensing unit 106 and/or implemented in the adhesive patch 108. For example, at least a portion of the physiological sensors may be integrated in the adhesive patch 108. As another example, at least a portion of the physiological sensors may be configured to be removably mounted onto the adhesive patch 108 (e.g., through being incorporated in or otherwise implemented via the cardiac sensing unit 106, which is configured to be removably mounted on to the adhesive patch 108 as discussed above).

In implementations, a number of ECG electrodes 202 may be embedded into the adhesive patch 108, as shown in FIG. 2. The ECG electrodes 202 are configured to sense one or more electrical signals indicative of ECG activity from a skin surface of the patient 100 (e.g., skin contacted directly or through a covering). The ECG electrodes 202 may be coupled to the cardiac sensing unit 106 by dedicated wiring within the adhesive patch 108, where the cardiac sensing unit 106 receives electrical signals sensed by the ECG electrodes 202 via the dedicated metal contacts or traces on the cardiac sensing unit 106 when the cardiac sensing unit 106 is mounted on the adhesive patch 108. Example ECG electrodes 202 include a metal electrode with an oxide coating, such as tantalum pentoxide electrodes. For instance, digital sensing electrodes can include skin-contacting electrode surfaces that may be deemed polarizable or non-polarizable depending on a variety of factors, including the metals and/or coatings used in constructing the electrode surface. All such electrodes can be used with the principles, techniques, devices, and systems described herein. As an illustration, the electrode surfaces can be based on stainless steel, noble metals such as platinum, or Ag—AgCl.

In examples, the ECG electrodes 202 can be used with an electrolytic gel dispersed between the electrode surface and the patient's skin. In other examples, the ECG electrodes 202 can be dry electrodes that do not need an electrolytic material or optionally can be used with an electrolytic material. For instances, such a dry electrode can be based on tantalum metal and have a tantalum pentoxide coating, as is described above. Such dry electrodes may be more comfortable for long-term monitoring applications, in various implementations.

In implementations, the ECG electrodes 202 can include additional components such as accelerometers, acoustic signal detecting devices, cardiovibrational sensors, or other measuring devices for additional parameters. For example, the ECG electrodes 202 may be configured to detect other types of physiological signals, such as thoracic fluid levels, heart vibrations, lung vibrations, respiration vibrations, patient movement, etc. Alternatively, or additionally, the cardiac sensing unit 106 and/or the adhesive patch 108 may include sensors or detectors separate from the ECG electrodes 202, such as separate motion detectors, bioacoustics sensors, cardiovibrational sensors, respiration sensors, temperature sensors, pressure sensors, and/or the like.

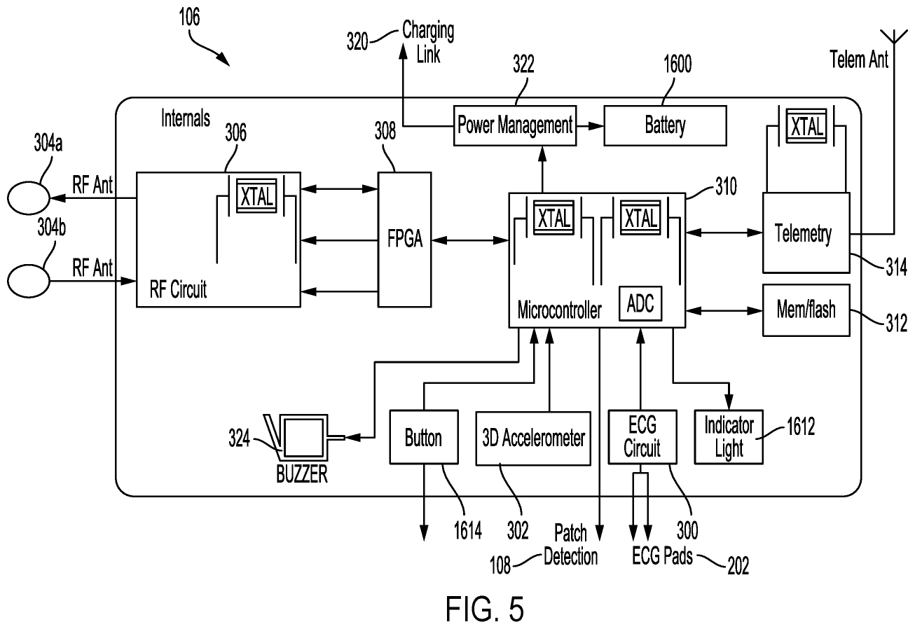

FIG. 5 illustrates an example electronic architecture for the cardiac sensing unit 106. The ECG circuit 300 may receive signals from the ECG electrodes 202 when the cardiac sensing unit 106 is attached to the adhesive patch 108, where the signals received from the ECG electrodes 202 include ECG waveforms sensed from the patient 100. In embodiments, the ECG electrodes 202 and cardiac sensing unit 106 may have a sampling rate in a range from about 250 Hz to about 500 Hz, from about 300 Hz to about 450 Hz, from about 350 Hz to about 400 Hz, including values and subranges therebetween. In embodiments, the cardiac sensing unit 106 may sample the signals from the ECG electrodes 202 after applying band-pass filtering by a 12 bit ADC. In implementations, during normal operation, the cardiac sensing unit 106 may transfer data from the ECG electrodes 202 to the remote server 104 “as-is,” where the remote server 104 uses the data for analysis as described in further detail below. Alternatively, in other implementations, the cardiac sensing unit 106 may perform at least some processing of the data from the ECG electrodes 202 (e.g., filtering of the data, analysis of the data, etc.). For example, the cardiac sensing unit 106 may generated digitized ECG signals from the data received from the ECG electrodes 202 and transmit the digitized ECG signals to the remote server 104 (e.g., directly or via the portable gateway 110).

In implementations, the wearable cardiac sensing device 102 can be designed to include a digital front-end where analog signals sensed by skin-contacting electrode surfaces of a set of digital sensing electrodes are converted to digital signals for processing. In this respect, the ECG circuit 300 may be an ECG digitizing circuit configured to provide digitized ECG signals of the patient 100 based on the electrical signals sensed from the patient 100. Typical wearable, ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device (e.g., the microcontroller 310 discussed below) for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

Internally, the wearable cardiac sensing device 102 includes a processor operationally coupled to, for example, the ECG circuit 300. In embodiments, the processor may be implemented as a microcontroller 310, as shown in FIG. 5. The microcontroller 310 stores instructions specifying how measurements (e.g., ECG, accelerometer, RF, etc.) are taken, how obtained data are transmitted, how to relay a status of the cardiac sensing unit 106, how/when the cardiac sensing unit 106 can enter a sleep level, and/or the like. In implementations, the instructions may specify the conditions for performing certain types of measurements. For example, the instructions may specify that a sensor of and/or connected to the cardiac sensing unit 106 may not commence measurements (e.g., ECG measurements, radiofrequency (RF) measurements, respiration rate measurements, and/or the like) unless the patient 100 is at rest or maintaining a certain posture. As another example, the instructions may identify the conditions that have to be fulfilled before any measurements can commence, such as a sufficient attachment level between the cardiac sensing unit 106 and the adhesive patch 108, or a sufficient attachment level between the adhesive patch 108 and the surface of the patient's body to which the adhesive patch 108 is attached. In implementations, the microcontroller 310 may have internal and/or external non-volatile memory banks (e.g., memory 312) that can be used for measurement directories and data, scheduler information, and/or a log of actions and errors. This non-volatile memory may allow for retaining data and status information in the case of a total power down. In implementations, instead of a microcontroller 310 as described above, the cardiac sensing unit 106 may include a microprocessor connected to a separate non-volatile memory, such as the memory 312.

The memory 312 may also be configured as a non-transitory memory configured to store data and/or signals of the cardiac sensing unit 106. For instance, the memory 312 may be configured to store digitized ECG signals of the patient 100.

The cardiac sensing unit 106 may further be able to establish wireless communications channels with other devices, such as the portable gateway 110 and/or the remote server 104, using a telemetry or wireless communications circuit 314. For example, the wireless communications circuit 314 may be a Bluetooth® unit. Additionally, or alternatively, the wireless communications circuit 314 may include other modules facilitating other types of wireless communication (e.g., Wi-Fi, cellular, etc.). The cardiac sensing unit 106 may transmit data to the remote server 104 using the wireless communications circuit 314. In implementations, the cardiac sensing unit 106 may transmit data indirectly to the remote server 104, such as by transmitting the signals and/or data to the portable gateway 110, with the portable gateway 110 transmitting the data received from the cardiac sensing unit 106 to the remote server 104. In implementations, the cardiac sensing unit 106 may instead transmit the data directly to the remote server 104.

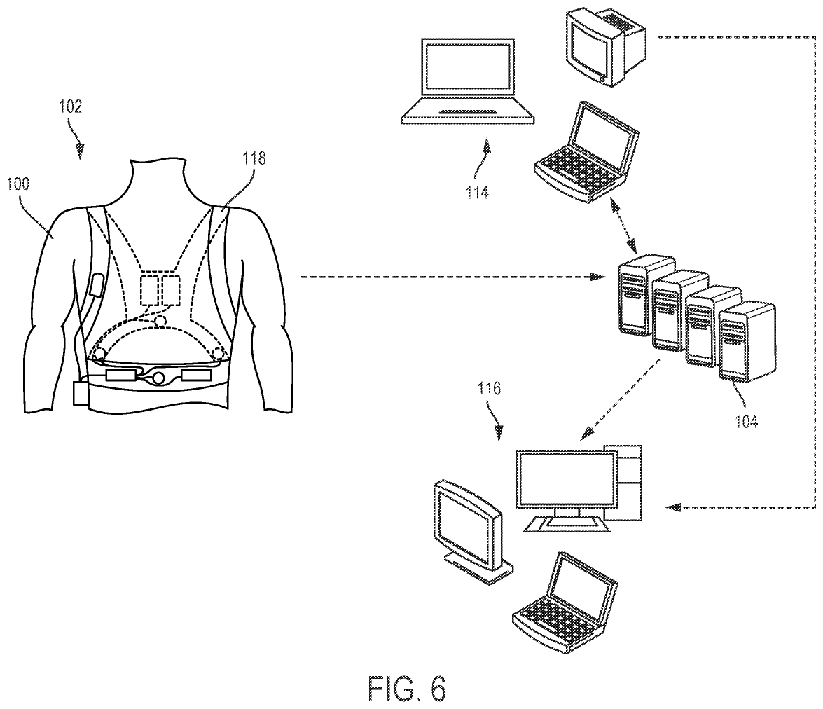

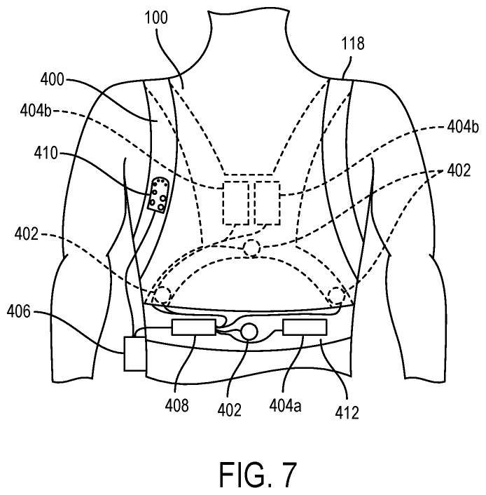

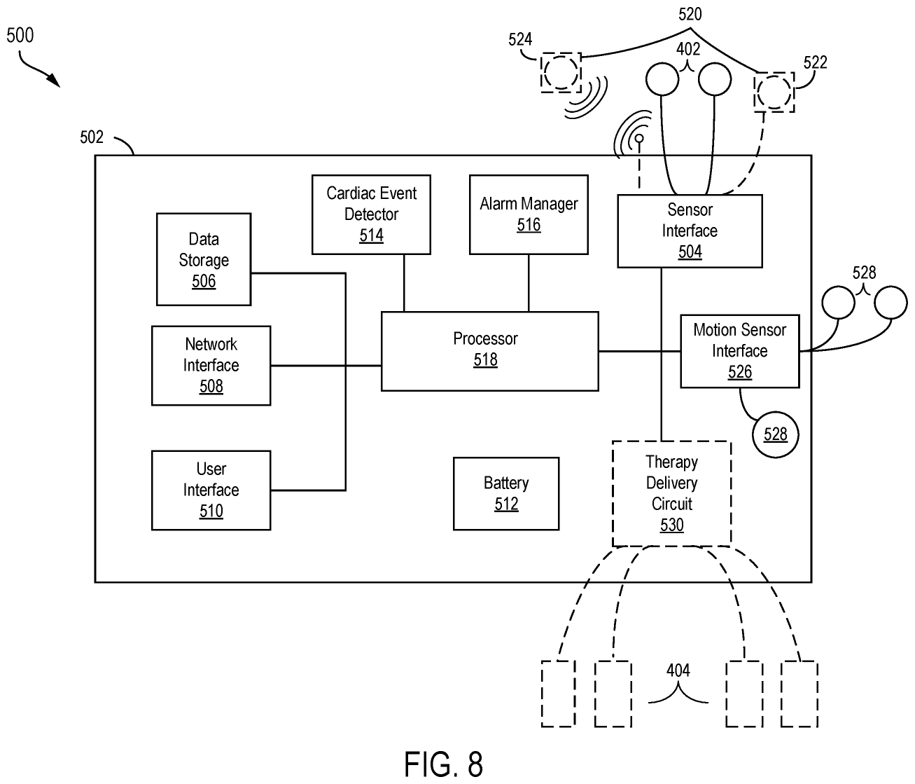

In implementations, the wearable cardiac sensing device 102 may be configured as a different wearable device from the example embodiments illustrated in FIGS. 1-5. For instance, FIG. 6 illustrates another example of a cardiac event monitoring system for improved ECG morphological analysis for determining abnormal cardiac events in a patient 100, according to implementations disclosed herein. The cardiac event monitoring system of FIG. 6 includes another embodiment of the wearable cardiac sensing device 102 configured to be bodily-attached to the patient 100. The wearable cardiac sensing device 102 according to FIG. 6 is a garment-based sensing device 118 configured to be worn about the patient's torso for an extended period of time, where various electronic components are temporarily and/or permanently mounted to the garment of the garment-based sensing device 118 (as described in further detail with respect to FIG. 7 below). Such a garment-based sensing device 118 can be, for example, capable of and designed for moving with the patient 100 as the patient 100 goes about their daily routine. For example, the garment-based sensing device 118 as described herein with respect to FIG. 6 (as well as FIGS. 7 and 8 below) can be a wearable cardioverter defibrillator, or wearable defibrillator, configured to be bodily-attached to the patient 100. In one example scenario, such wearable defibrillators can be worn nearly continuously or substantially continuously for a week, two weeks, a month, or two or three months at a time. During the period of time in which it is worn by the patient 100, the wearable defibrillator can be configured to continuously or substantially continuously monitor the vital signs of the patient 100 and, upon determining that the patient 100 is experiencing a treatable arrhythmia, deliver one or more therapeutic electrical pulses to the patient 100. For example, such therapeutic shocks can be pacing, defibrillation, cardioversion, or transcutaneous electrical nerve stimulation (TENS) pulses.

The cardiac event monitoring system shown in FIG. 6 (including the remote server 104, technician interface(s) 114, and caregiver interface(s) 116) may generally function similarly to the cardiac event monitoring system shown and described above with respect to FIG. 1. Similar to the example wearable cardiac sensing device 102 shown in FIG. 2, the example wearable cardiac sensing device 102 shown in FIG. 6 is configured for long-term and/or extended use or wear by, or attachment or connection to, the patient 100, as discussed above. Such substantially or nearly continuous use, monitoring, or wear similar to the usages described above may nonetheless be considered continuous use, monitoring, or wear. In implementations, the example wearable cardiac sensing device 102 may also include a portable gateway, similar to the portable gateway 110 shown and described above, where the portable gateway facilitates communication between the garment-based sensing device 118 and the remote server 104. The portable gateway 110 may, additionally or alternatively, may be configured to perform some or all of the analysis described below.

FIG. 7 illustrates the garment-based sensing device 118 in more detail, according to various implementations. As shown in FIG. 7, the garment-based sensing device 118 can include one or more of the following: a garment 400 configured to be worn around the patient's torso, a plurality of physiological sensors, one or more therapy electrodes 404a and 404b (collectively referred to herein as therapy electrodes 404), a medical device controller 406, a connection pod 408, a patient interface pod 410, a belt 412, or any combination of these components. In examples, at least some of the components of the garment-based sensing device 118 (e.g., at least a portion of the physiological sensors, including the ECG electrodes 402; at least a portion of the therapy electrodes 404; etc.) are configured to be mounted on or affixed to the garment 400, such as by mating hooks, hook-and-loop fabric strips, receptacles (e.g., pockets), and the like. For example, the ECG electrodes 402 may be mounted on the garment 400 by hook-and-loop fabric strips on the electrodes 402 and the garment 400, and the therapy electrodes 404 may be mounted on the garment 400 by being inserted into receptacles of the garment 400 (e.g., into pockets of the garment 400). In examples, at least some of the components of the garment-based sensing device 118 (e.g., at least a portion of the physiological sensors, including the ECG electrodes 402; at least a portion of the therapy electrodes 404; etc.) can be permanently integrated into the garment 400, such as being sewn into or permanently adhered onto the garment 400. In examples, at least some of the components may be connected to each other through external cables, through sewn-in connections (e.g., wires woven into the fabric of the garment 400 or between layers of the garment 400), through conductive fabric of the garment 400, and/or the like.

The medical device controller 406 can be operatively coupled to the ECG electrodes 402, which can be affixed to the garment 400 (e.g., assembled into the garment 400 or removably attached to the garment 400, for example, using hook-and-loop fasteners) or permanently integrated into the garment 400. In implementations, the medical device controller 406 is also operatively coupled to the therapy electrodes 404. The therapy electrodes 404 may be similarly assembled into the garment 400 (e.g., into pockets or other receptacles of the garment 400) or permanently integrated into the garment 400. As shown in FIG. 4, the ECG electrodes 402 and/or the therapy electrodes 404 can be directly operatively coupled to the medical device controller 406 through the connection pod 408. Component configurations other than those shown in FIG. 7 are also possible. For example, the ECG electrodes 402 can be configured to be attached at various positions about the body of the patient 100. In implementations, at least one of the ECG electrodes 402 and/or at least one of the therapy electrodes 404 can be included on a single integrated patch and adhesively applied to the patient's body. In implementations, at least one of the ECG electrodes 402 and/or at least one of the therapy electrodes 404 can be included in multiple patches and adhesively applied to the patient's body. Such patches may be in wired (e.g., directly or via the connection pod 408) or wireless connection with the medical device controller 406. Similar implementations may also be extended to the non-ECG physiological sensors of the garment-based sensing device 118.

The ECG electrodes 402 are configured to detect one or more cardiac signals, such as electrical signals indicative of ECG activity from a skin surface of the patient 100. In implementations, the ECG electrodes 402 of the garment-based sensing device 118 may be configured similarly to the ECG electrodes 202 of the adhesive patch 108 described above. In implementations, the therapy electrodes 404 can also be configured to include sensors that detect ECG signals as well as, or in the alternative from, other physiological signals from the patient 100. The connection pod 408 can, in various examples, include a signal processor configured to amplify, filter, and digitize these cardiac signals prior to transmitting the cardiac signals to the medical device controller 406.

Additionally, the therapy electrodes 404 can be configured to deliver one or more therapeutic cardioversion/defibrillation shocks to the body of the patient 100 when the medical device controller 406 determines that such treatment is warranted based on the signals detected by the ECG electrodes 402 and processed by the medical device controller 406. Example therapy electrodes 404 can include conductive metal electrodes such as stainless-steel electrodes. In implementations, the therapy electrodes 404 may also include one or more conductive gel deployment devices configured to deliver conductive gel between the metal electrode and the patient's skin prior to delivery of a therapeutic shock.

In implementations, the medical device controller 406 may also be configured to warn the patient 100 prior to the delivery of a therapeutic shock, such as via output devices integrated into or connected to the medical device controller 406, the connection pod 408, and/or the patient interface pod 410. The warning may be auditory (e.g., a siren alarm, a voice instruction indicating that the patient 100 is going to be shocked), visual (e.g., flashing lights on the medical device controller 406), haptic (e.g., a tactile, buzzing alarm generated by the connection pod 408), and/or the like. If the patient 100 is still conscious, the patient 100 may be able to delay or stop the delivery of the therapeutic shock. For example, the patient 100 may press one or more buttons on the patient interface pod 410 and/or the medical device controller 406 to indicate that the patient 100 is still conscious. In response to the patient 100 pushing the one or more buttons, the medical device controller 406 may delay or stop the delivery of the therapeutic shock.

In implementations, a garment-based sensing device 118 as described herein can be configured to switch between a therapeutic mode and a monitoring mode such that, when in the monitoring mode, the garment-based sensing device 118 is configured to only monitor the patient 100 (e.g., not provide or perform any therapeutic functions). For example, in such implementations, therapeutic components such as the therapy electrodes 404 and associated circuitry may be decoupled from (or coupled to) or switch out of (or switched into) the garment-based sensing device 118. As an illustration, a garment-based sensing device 118 can have optional therapeutic elements (e.g., defibrillation and/or pacing electrode components and associated circuitry) that are configured to operate in a therapeutic mode. The optional therapeutic elements may be physically decoupled from the garment-based sensing device 118 as a means to convert the garment-based sensing device 118 from a therapeutic mode into a monitoring mode. Alternatively, the optional therapeutic elements may be deactivated (e.g., by means of a physical or software switch), essentially rendering the garment-based sensing device 118 as a monitoring-only device for a specific physiological purpose for the particular patient 100. As an example of a software switch, an authorized person may be able to access a protected user interface of the garment-based sensing device 118 and select a preconfigured option or perform some other user action via the user interface to deactivate the therapeutic elements of the garment-based sensing device 118.

FIG. 8 illustrates a sample component-level view of a medical device controller 500 included in a garment-based sensing device 118. The medical device controller 500 is an example of the medical device controller 406 shown in FIG. 7 and described above. As shown in FIG. 8, the medical device controller 500 may include a housing 502 configured to house a number of electronic components, including a sensor interface 504, a data storage 506, a network interface 508, a user interface 510, at least one battery 512 (e.g., positioned within a battery chamber configured for such a purpose), a cardiac event detector 514, an alarm manager 516, at least one processor 518, and a therapy delivery circuit 530. As described above, in some implementations, the garment-based sensing device 118 may include like components to those described above but may not include therapeutic components. That is, in some implementations, the garment-based sensing device 118 can include ECG monitoring components and not provide therapy to the patient 100. Accordingly, the garment-based sensing device 118 may not include the therapy delivery circuit 530 and the therapy electrodes 404 (shown in dotted lines), or the therapy delivery circuit 530 and the therapy electrodes 404 may be disconnectable from the rest of the garment-based sensing device 118.

In implementations, the processor 518 includes one or more processors (or one or more processor cores) that are each configured to perform a series of instructions that result in the manipulation of data and/or the control of the operation of the other components of the medical device controller 500. In implementations, when executing a specific process (e.g., monitoring sensed electrical data of the patient 100), the processor 518 can be configured to make specific logic-based determinations based on input data received. The processor 518 may be further configured to provide one or more outputs that can be used to control or otherwise inform subsequent processing to be carried out by the processor 518 and/or other processors or circuitry to which the processor 518 is communicably coupled. Thus, the processor 518 reacts to a specific input stimulus in a specific way and generates a corresponding output based on that input stimulus. In example cases, the processor 518 can proceed through a sequence of logical transitions in which various internal register states and/or other bit cell states internal or external to the processor 518 may be set to logic high or logic low.

As referred to herein, the processor 518 can be configured to execute a function where software is stored in a data store (e.g., the data storage 506) coupled to the processor 518, the software being configured to cause the processor 518 to proceed through a sequence of various logic decisions that result in the function being executed. The various components that are described herein as being executable by the processor 518 can be implemented in various forms of specialized hardware, software, or a combination thereof. For example, the processor 518 can be a digital signal processor (DSP) such as a 24-bit DSP processor. As another example, the processor 518 can be a multi-core processor, e.g., having two or more processing cores. As another example, the processor 518 can be an Advanced RISC Machine (ARM) processor, such as a 32-bit ARM processor. The processor 518 can execute an embedded operating system and further execute services provided by the operating system, where these services can be used for file system manipulation, display and audio generation, basic networking, firewalling, data encryption, communications, and/or the like.

The data storage 506 can include one or more of non-transitory media, such as flash memory, solid state memory, magnetic memory, optical memory, cache memory, combinations thereof, and others. The data storage 506 can be configured to store executable instructions and data used for operation of the medical device controller 500. In implementations, the data storage 506 can include sequences of executable instructions that, when executed, are configured to cause the processor 518 to perform one or more functions. Additionally, the data storage 506 can be configured to store information such as digitized ECG signals of the patient 100.

In examples, the network interface 508 can facilitate the communication of information between the medical device controller 500 and one or more devices or entities over a communications network. For example, the network interface 508 can be configured to communicate with the remote server 104 or other similar computing device. Using the network interface 508, the garment-based sensing device 118 may transmit, for example, ECG signals, other physiological signals, indications of abnormal cardiac events, etc., to the remote server 104. In implementations, the network interface 508 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the garment-based sensing device 118, such as a device similar to the portable gateway 110). The intermediary device(s) may in turn communicate the data to the remote server 104 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 104 over a Wi-Fi communications link based on the IEEE 802.11 standard. In implementations, the network interface 508 may be configured to instead communicate directly with the remote server 104 without the use of intermediary device(s). In such implementations, the network interface 508 may use any of the communications links and/or protocols provided above to communicate directly with the remote server 104.

The sensor interface 504 can include physiological signal circuitry that is coupled to one or more externally applied sensors 520. The externally applied sensors 520 may include, for example, one or more externally applied physiological sensors. As shown, the sensors may be coupled to the medical device controller 500 via a wired or wireless connection. The externally applied sensors 520 may include the ECG electrodes 402 configured to sense one or more electrical signals indicative of ECG activity from the skin surface of the patient 100, as well as one or more non-ECG sensors such as a cardiovibration sensor 522 and a tissue fluid monitor 524 (e.g., configured similarly to the thoracic fluid sensor implemented through the at least one RF antenna 304a, 304b and RF circuitry 306 discussed in further detail below with reference to FIG. 5). In implementations, the sensor interface 504 and/or the processor 518 may be configured to provide digitized ECG signals of the patient 100 based on electrical signals sensed by the ECG electrodes 402. In this sense, the sensor interface 504 and/or the processor 518 may be considered an ECG digitizing circuit. The digitized ECG signals of the patient 100 may be stored in the data storage 506.

In implementations, the cardiac event detector 514 can be configured to monitor the patient's ECG signal for an occurrence of a cardiac event such as an arrhythmia or other similar cardiac event. The cardiac event detector 514 can be configured to operate in concert with the processor 518 to execute one or more methods that process received ECG signals from, for example, the ECG sensing electrodes 202 and determine the likelihood that the patient 100 is experiencing a cardiac event, such as a treatable arrhythmia. The cardiac event detector 514 can be implemented using hardware or a combination of hardware and software. For instance, in some examples, the cardiac event detector 514 can be implemented as a software component that is stored within the data storage 506 and executed by the processor 518. In this example, the instructions included in the cardiac event detector 514 can cause the processor 518 to perform one or more methods for analyzing a received ECG signal to determine whether an adverse cardiac event is occurring, such as a treatable arrhythmia. In other examples, the cardiac event detector 514 can be an application-specific integrated circuit (ASIC) that is coupled to the processor 518 and configured to monitor ECG signals for adverse cardiac event occurrences. Thus, examples of the cardiac event detector 514 are not limited to a particular hardware or software implementation.

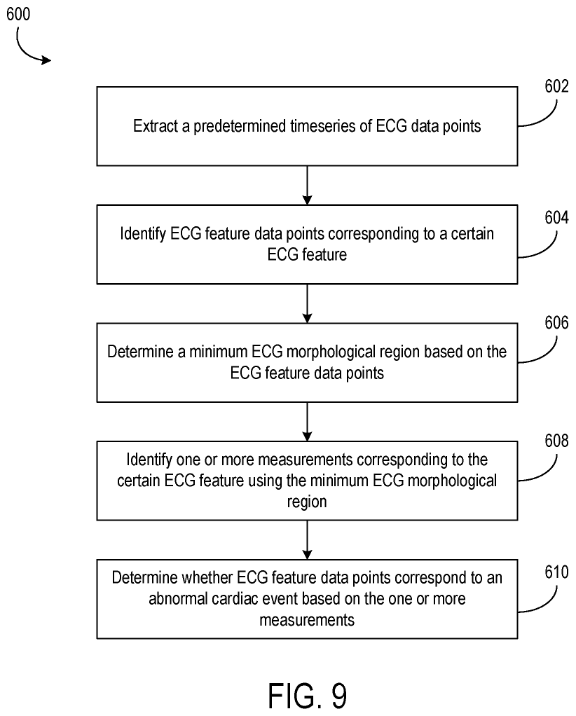

Regardless of the embodiment of the wearable cardiac sensing device 102 used, a processor in communication with the wearable cardiac sensing device 102 may be configured to process ECG data gathered by the wearable cardiac sensing device 102 to determine abnormal cardiac events in the patient 100. Accordingly, FIG. 9 illustrates a sample process flow for applying improved ECG morphological analysis for determining abnormal cardiac events in a patient. The sample process 600 shown in FIG. 9 can be implemented by a processor in communication with the wearable cardiac sensing device 102. In implementations, the processor may be part of the wearable cardiac sensing device 102. For example, where the wearable cardiac sensing device 102 includes a cardiac sensing unit 106 and a portable gateway 110, the processor may be implemented by the microcontroller 310 of the cardiac sensing unit 106. As another example, the processor may be implemented in the portable gateway 110. As another example, where the wearable cardiac sensing device 102 includes a garment-based sensing device 118, the processor may be implemented by the processor 518 (e.g., in conjunction with the cardiac event detector 514). As another example, the processor may be implemented in the remote server 104. As another example, the processor may be implemented partially by a processor at the wearable cardiac sensing device 102 and partially by a processor at the remote server 104.

The processor is configured to extract a predetermined timeseries of ECG data points at step 602. In implementations, as described above, digitized ECG signals of the patient 100 may be stored in a non-transitory memory of the wearable cardiac sensing device 102. As such, the processor may receive the digitized ECG signals from the wearable cardiac sensing device 102 (e.g., via a transmission of the wearable cardiac sensing device 102) or retrieve the digitized ECG signals from the non-transitory memory of the wearable cardiac sensing device 102. For example, the processor may receive or retrieve a predetermined timeseries of ECG data points as the data points forming a segment of the digitized ECG signals, where each of the data points represents an ECG amplitude recorded by the wearable cardiac sensing device 102 at a given time. The digitized ECG signal segments may be pre-segmented (e.g., by the wearable cardiac sensing device 102, such as at the cardiac sensing unit 106, at the portable gateway 110, or at the controller 500 of the garment-based sensing device 118). Alternatively, the digitized ECG signal segments may be segmented by the processor, for instance, at the time the processor receives or retrieves the digitized ECG signals. The segments may be generated according to a predetermined time period (e.g., 60 seconds of ECG data, 90 seconds of ECG data, 120 seconds of ECG data, 5 minutes of ECG data, and/or the like), according to a transmission schedule (e.g., segmented to facilitate transmission of the digitized ECG signals to the remote server 104), according to a symptom event recorded by the wearable cardiac sensing device 102, such that each segment represents a single ECG cycle, and/or the like.

In implementations, the ECG timeseries can be based on a predetermined sampling frequency and predetermined gain. For example, the predetermined sampling frequency can be between about 100 Hz to about 1000 Hz. As an example, the predetermined sampling frequency can be 150 Hz, 240 Hz, 300 Hz, 260 Hz, or 480 Hz. For example, the predetermined gain can be between about 50 and about 1000. As an example, the predetermined gain can be 120, 180, 200, 250, 300, or 350. In implementations, ECG can be digitized in a ECG pre-processor configuration (e.g., ECG acquisition circuitry) prior to being processed by the processes described in connection with FIG. 9. In implementations, for example, the ECG data can be in the form of a single ECG channel, or a plurality of ECG channels (e.g., two or more ECG channels). As another example, implementations, there can be two 16 bit ECG channels that can be acquired and sent to a digital signal processor for signal processing. In examples, a sampling rate can be dynamic or predetermined, e.g., based on a rate determined by the digital signal processor. In examples, an ECG streaming command can be issued by a processor to initiate, pause, or stop the acquisition and streaming of ECG samples from the ECG acquisition circuitry to the digital signal processor. In examples, an acknowledge frame can be provided to allow for synchronization of the ECG data streamed from the ECG acquisition circuitry and the digital signal processor.

In some examples, an electrode falloff signal or indication can be included in the transmission frame, e.g., to allow for the digital signal processor to analyze and determine whether an ECG electrode is deemed to not be making proper contact with skin of the patient. For example, the ECG sensing interface of the processor can incorporate a falloff detection circuit. In this example, a low level alternating current (AC) signal can be applied to the body of the patient and sensed by each ECG electrode circuit. For instance, this fall off signal can be digitized at a 1 Hz interval.

The processor may identify ECG feature data points corresponding to a certain ECG feature from within the digitized ECG signals of the patient 100 at step 604. Example ECG features may include the P wave, the QRS complex including the QR segment and RS segment, and the T wave. In implementations, the processor may identify the features of an ECG signal by applying a feature extractor. For example, the processor may implement a rhythm classifier stored in a non-transitory computer readable medium (e.g., a memory, a programmable circuit board, a field programmable gate array, an integrated circuit, any combination thereof, and/or the like). The rhythm classifier may include at least one neural network trained based on a historical collection of ECG signal portions with known rhythm information. Among the rhythm information the rhythm classifier is trained to identify may be the certain ECG feature. Additionally, the processor may detect time data corresponding to the certain ECG feature.

As an illustration, in implementations, the processor may use a Pan-Tompkins-based QRS detector. The processor may first filter the ECG feature data points. For instance, the processor may receive or retrieve raw ECG data sampled at 250 Hz. The processor may then remove baseline wander, high frequency noise, and 50/60 Hz interference. This filtering may be represented mathematically by denoting the raw ECG data as x and representing the process as:

y₁=bpf*x

In this equation, bpf represents band pass filtering, * stands for convolution, and y₁ represents the filtered signal. The processor may find the derivative of the filtered y₁ signal shown above square the result, which may be represented as:

$${y}_{2} = {( \frac{{dy}_{1}}{dt} )}^{2}$$

Next, the processor may apply a moving average to the y₂ result, which may be further represented mathematically by:

y₃=Moving AverageFilter*y₂

The processor then may apply an adaptive power threshold to locate the QRS complexes in the y₃ signal. However, as other illustrations, the processor may use a Hilbert transform process or a phasor transform process to identify the ECG feature data points corresponding to the certain ECG feature. In implementations, the rhythm classifier may determine a confidence score associated with the detected ECG feature data points (e.g., output a confidence score associated with the probability that the certain ECG feature data points were identified correctly).

The processor determines a minimum ECG morphological region based on the ECG feature data points at step 606. The minimum ECG morphological region may be a minimum region where all or a predetermined number, percentage, fit, etc. of the ECG feature data points are located within and/or on the boundaries of the region. For example, in implementations, the minimum ECG morphological region may be configured such that all of the ECG feature data points are located within and/or on the boundaries of the minimum ECG morphological region. In implementations, the minimum ECG morphological region may be configured such that a predetermined number of the ECG feature data points are located within and/or on the boundaries of the minimum ECG morphological region, such as at least 80% to 100% of the ECG feature data points (e.g., at least 80%, at least 85%, at least 90%, at least 95%, at least 98%, etc.). In implementations, the minimum ECG morphological region may be configured as a best fit region for the ECG feature data points, with the predetermined number of ECG feature data points enclosed within or on the ECG morphological region controlled by the best fit process.

In implementations, the minimum ECG morphological region may be a minimum convex ECG morphological region. To illustrate, in implementations, the minimum ECG morphological region may be a polygon where each of the ECG feature data points are located within the polygon or at one of the vertices of the polygon. For example, the minimum ECG morphological region may be structured such that each of the ECG feature data points is disposed at either a plurality of convex vertices or within an enclosed region formed by a plurality of line segments interconnecting the plurality of convex vertices. As another example, the minimum ECG morphological region may be structured such that each of the ECG feature data points is disposed at either a plurality of vertices, where at least one of the plurality of vertices is outward-facing (e.g., with respect to the interior of the minimum ECG morphological region), or within an enclosed region formed by a plurality of line segments interconnecting the plurality of outward-facing vertices. In instances, each of the plurality of vertices may be outward facing. In instances, at least three of the plurality of vertices may be outward facing.

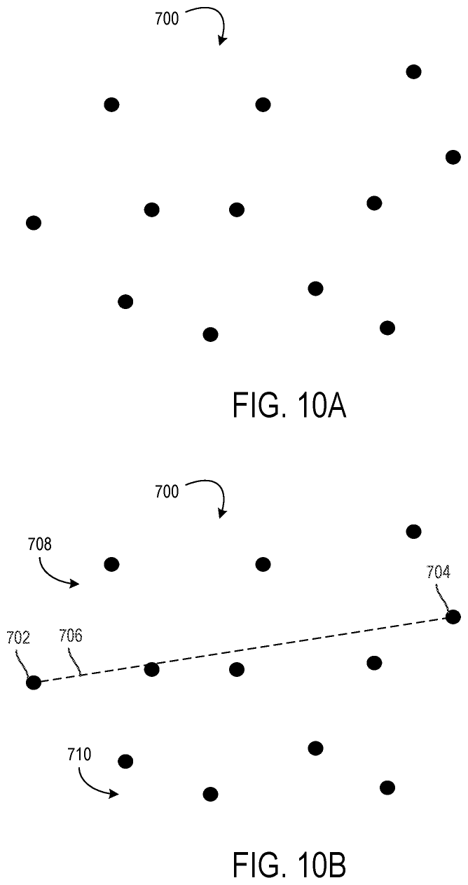

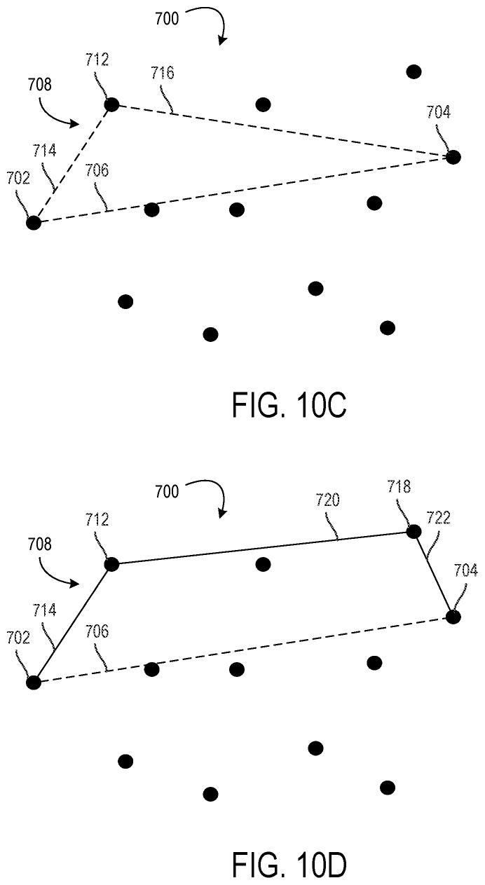

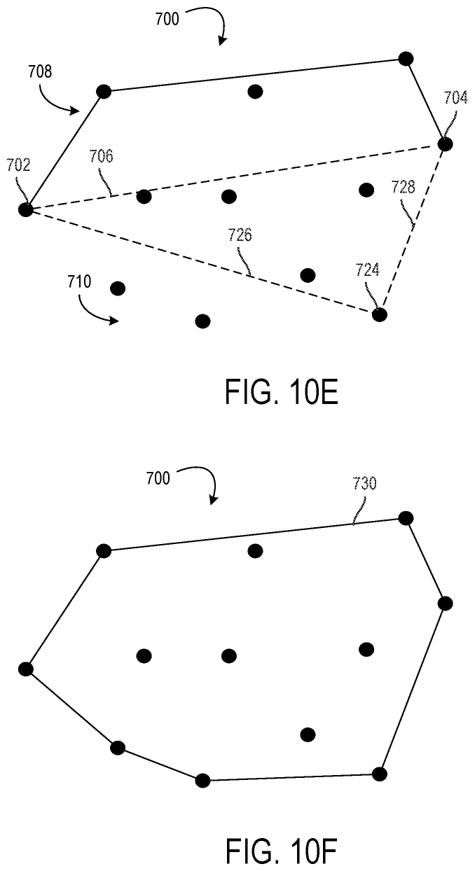

FIGS. 10A-10F illustrate an example of how a processor can form a minimum morphological region (e.g., using convex hull methods). FIG. 10A shows a sample set of data points 700. To create the minimum morphological region, the processor first identifies the leftmost point 702 and the rightmost point 704, as shown in FIG. 10B. The processor uses the leftmost point 702 and the rightmost point 704 to divide the data points 700 into two groups using a dividing line 706: a left data point subgroup 708 that is left of the dividing line 706 (or right of the dividing line 706 from leftmost point 702 to rightmost point 704) and a right data point subgroup 710 that is right of the dividing line 706 (or right of the dividing line 706 from rightmost point 704 to leftmost point 702). As illustrated in FIG. 10C, for the left data point subgroup 708, the processor next determines a point 712 that is furthest from the dividing line 706. For instance, the processor may identify the furthest point 712 by drawing a line perpendicular to the dividing line 706 to each of the points in the left data point subgroup 708 and determine which of these perpendicular lines is the longest. The processor then draws a first line 714 between the leftmost point 702 and the furthest point 712 and a second line 716 between the rightmost point 704 and the furthest point 712.

The processor then repeats this enclosing process for the first line 714 and the second line 716, ignoring any of the left data points subgroup 708 that is already bounded by the dividing line 706, first line 714, and second line 716. The processor continues repeating this enclosing process until all of the left data point subgroup 708 is either a vertex of the resulting polygon or enclosed within the resulting polygon. Continuing with the example shown in FIG. 10C, there are no data points 708 beyond the first line 714. Therefore, the processor may determine that the first line 714 is one of the bounding segments of the minimum morphological region. For the second line 716, point 718 is the furthest point from the second line 716. As illustrated in FIG. 10D, drawing a third line 720 between point 712 and point 718 and a fourth line 722 between point 718 and point 704 creates a polygon (e.g., formed from lines 714, 720, and 722 and the dividing line 706) where all of the left data points subgroup are either a vertex of the polygon (e.g., points 702, 712, 718, and 704) or bounded within the polygon. As such, the processor concludes this process for the left data point subgroup 708.

The processor also repeats the enclosing process for the right data points subgroup 710, as shown in FIG. 10E. Accordingly, the processor determines that point 724 is furthest from the dividing line 706 within the right data points subgroup 710. The processor draws a fifth line 726 between the furthest point 724 and the leftmost point 702 and a sixth line 728 between the furthest point 724 and the rightmost point 704. The processor continues repeating the enclosing process until all of the points of the right data points subgroup 710 are a vertex of the resulting polygon or enclosed within the resulting polygon. FIG. 10F illustrates the resulting minimum enclosing region 730. As shown in FIG. 10F, all of the data points 700 are either bound within the minimum enclosing region 730 or a convex vertex of the minimum enclosing region 730.

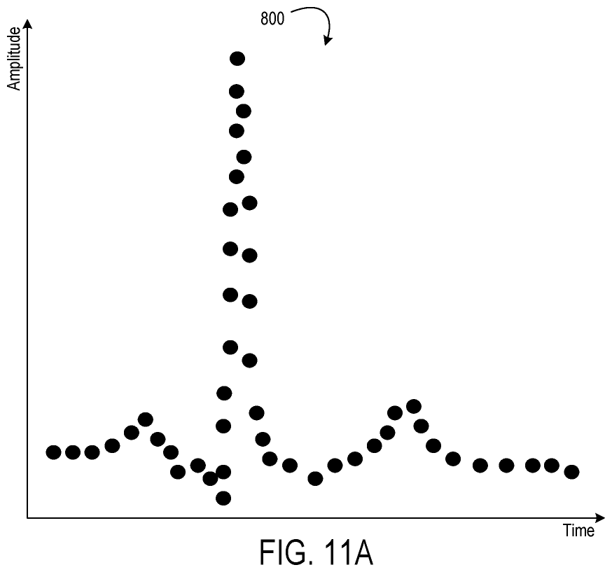

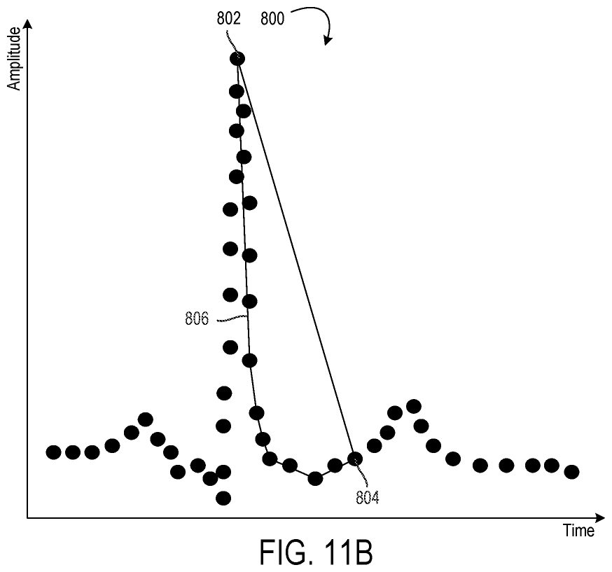

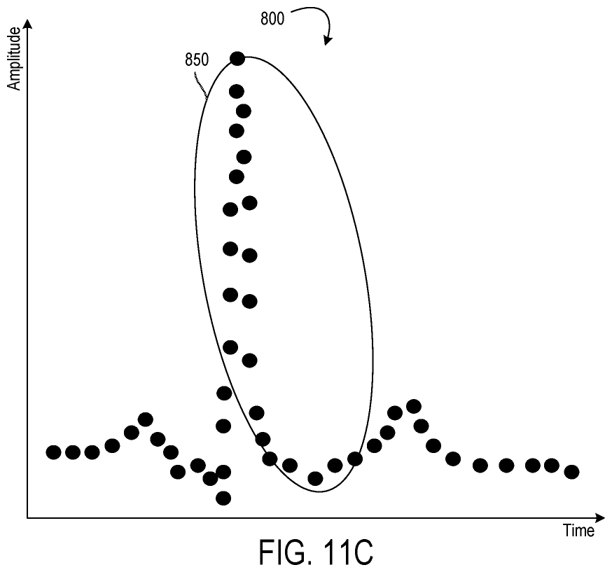

A similar process of forming a minimum enclosing region may be applied to the ECG feature data points as part of performing step 606. FIG. 11A shows an example of ECG data points 800 (e.g., data points generated by the wearable cardiac sensing device 102 of ECG amplitude measured at a given time) for a single cardiac cycle. In the example of FIGS. 11A and 11B, the ECG feature data points corresponding to the certain ECG feature (e.g., according to step 604) are a subset of the ECG data points corresponding to an RS segment of the cardiac cycle. As shown in FIG. 11B, the processor connects a first point 802 of the RS segment and a last point 804 of the RS segment. The processor then proceeds to apply the enclosing process to the subset of ECG data points corresponding to the RS segment to form the minimum ECG morphological region 806 illustrated in FIG. 11B.

However, other methods may be used to generate a minimum ECG morphological region at step 606. For instance, a minimum ECG morphological region may not be entirely convex. Instead, the minimum ECG morphological region may be generated similarly to the methods described above with respect to FIGS. 10A-11B but modified to have sharp or rounded corners at or around the ECG feature data points at the edges of the ECG feature, such as ECG feature data points a predetermined distance from the centroid or center line (e.g., similar to line 706) of the ECG feature. As another example, a minimum ECG morphological region may be constructed by using polynomial splines to draw smooth polygons around the ECG feature data points.

As another example, a minimum ECG morphological region may be an ellipse. An example of an minimum ECG morphological region 850 formed as an ellipse is shown in FIG. 11C. In implementations, the processor may determine a minimum ECG morphological region configured as an ellipse using a direct linear least squares approach. For example, the generic equation for a conic section with linear parameters a, b, c, d, e, and f can be represented as the following:

$$F ( x , y ) = {ax}^{2} + bxy + {cy}^{2} + dx + ey + f = 0$$

The polynomial F(x, y) can be considered the algebraic difference of any point (x, y) from the conic section. Thus, the following formula can be written as the sum of the squares of the algebraic distances from the conic section:

$$d ( a ) = {\sum }_{i = 1}^{N} {F ( {x}_{i} )}^{2} , where a = {[ a , b , c , d , e , f ]}^{T}$$

To ensure that the conic section best fitted to the set of points is an ellipse (e.g., instead of a hyperbola), a can be scaled to impose the constraint 4ac−b²=1. A least squares approach may then be used with the above formulas to identify the ellipse that best fits the data points. For example, a least squares approach can be used to minimize ∥Da∥² subject to the constraint a^{T}Ca=1. In this formulation, the design matrix representing the minimization of F is:

$$D = ( \begin{matrix}{x}_{1}^{2} & {x}_{1} {y}_{1} & {y}_{1}^{2} & {x}_{1} & {y}_{1} & 1 \\ {x}_{2}^{2} & {x}_{2} {y}_{2} & {y}_{2}^{2} & {x}_{2} & {y}_{2} & 1 \\ ⋮ & ⋮ & ⋮ & ⋮ & ⋮ & ⋮ \\ {x}_{n}^{2} & {x}_{n} {y}_{n} & {y}_{n}^{2} & {x}_{n} & {y}_{n} & 1\end{matrix} )$$

The constraint matrix, representing the constraint a^{T}Ca=1, is:

$$C = ( \begin{matrix}0 & 0 & 2 & 0 & 0 & 0 \\ 0 & - 1 & 0 & 0 & 0 & 0 \\ 2 & 0 & 0 & 0 & 0 & 0 \\ 0 & 0 & 0 & 0 & 0 & 0 \\ 0 & 0 & 0 & 0 & 0 & 0 \\ 0 & 0 & 0 & 0 & 0 & 0\end{matrix} )$$

The solution with the smallest positive eigenvalue and corresponding eigenvector represents the best fit ellipse to the data points.

However, an ellipse where a predetermined number or percentage of the ECG feature data points being on the ellipse and/or enclosed within the ellipse may be desired in implementations, while a least squares approach may be variable in this regard. In implementations, the processor may determine an ellipse with the smallest area where all or a predetermined number of the ECG feature data points are either on the ellipse or enclosed within the ellipse. For example, the processor may use the Mahalanobis distance for the ECG feature data points to identify an ellipse where all or a predetermined number of the ECG feature data points are on the ellipse and/or enclosed within the ellipse. Generally, the centroid of the ECG feature data points may be determined, and the Mahalanobis distance may be used to find the distance of a point from the centroid on an ellipse. If, for instance, the processor determines the Mahalanobis distance for all of the ECG feature data points, the data point with the largest Mahalanobis distance would be correlated with an ellipse where all or most of the ECG feature data points are located on the ellipse and/or enclosed within the ellipse. Alternatively, the processor may determine the Mahalanobis distance for all of the ECG feature data points and further determine, for each corresponding ellipse, whether each of the ECG feature data points is located within and/or on the ellipse. The processor may determine that the ellipse with the smallest area and with a predetermined number of data points located within and/or on the ellipse as the minimum ECG morphological region.

As another example, the processor may determine the minimum ECG morphological region by applying morphological closing. Morphological closing may be applied by enclosing the ECG feature data set, such as by enclosing the ECG feature data set as a trace, as described below. Then, resulting polygon can be dilated, followed by an erosion, to produce a morphologically closed polygon representing the minimum ECG morphological region.

Returning to the process flow 600 shown in FIG. 9, the processor identifies one or more measurements corresponding to the certain ECG feature using the minimum ECG morphological region at step 608. For example, the processor may determine an area of the minimum ECG morphological region. As another example, the processor may determine a trace of the ECG feature data points by defining a polygon constructed by connecting succeeding points in the time series. The processor may form a signal trace region by adding a final enclosing line segment formed by connecting the first and last points in the ECG feature data points. Alternatively, the processor may determine a signal trace region of the ECG feature data points by adding points on the isoelectric line corresponding to the ECG feature data points and connecting the first and last of the ECG feature data points to the isoelectric line to form the region. The processor may then measure the ratio of the perimeter of the convex hull region to the perimeter of the region formed by the signal trace. As another example, the processor may determine the perimeter of the minimum ECG morphological region. As another example, if the minimum ECG morphological region is an ellipse, the processor may determine one or more measurements corresponding to the ellipse, such as the center, the foci, the major axis, the minor axis, the roundness, and/or the like.

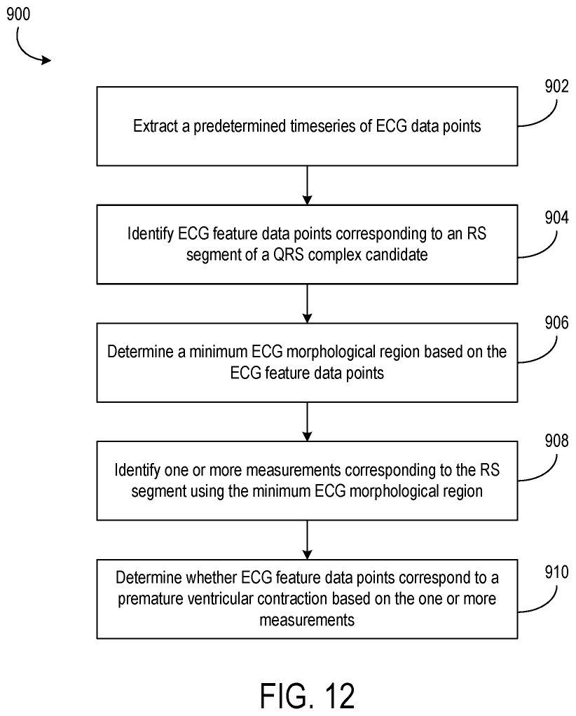

Finally, the processor may determine whether the ECG feature data points correspond to an abnormal cardiac event based on the one or more measurements at step 610. For example, the processor may use an area of the minimum ECG morphological region corresponding to an RS segment to determine whether the cardiac cycle represents a premature ventricular contraction (PVC). As another example, the processor may compare an area of the minimum ECG morphological region corresponding to a QRS complex to the area of the trace of the QRS complex to determine whether the QRS complex contains a notch. These examples are discussed in further detail below with respect to FIGS. 12-17.

FIG. 12 illustrates a sample process flow for applying improved ECG morphological analysis for identifying PVCs in a patient. Similar to the sample process 600, the sample process 900 shown in FIG. 12 can be implemented by a processor in communication with the wearable cardiac sensing device 102. The processor may be implemented at the wearable cardiac sensing device 102 (e.g., at the cardiac sensing unit 106, portable gateway 110, or garment-based sensing device 118) and/or at the remote server 104.

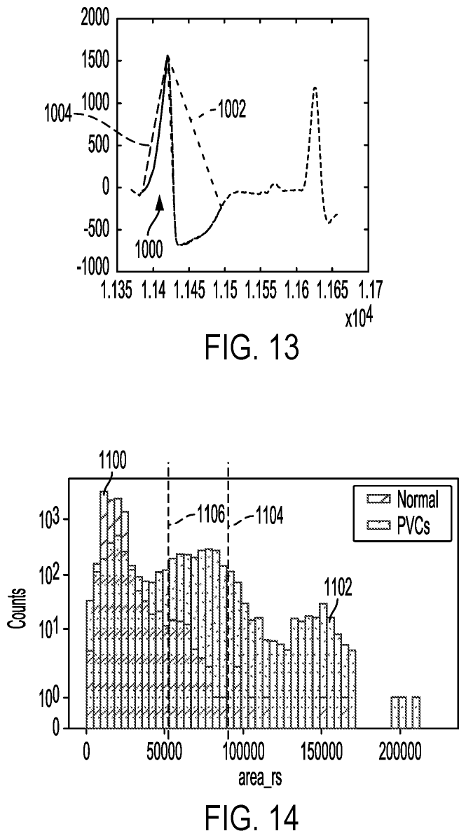

The processor extracts a predetermined timeseries of ECG data points at step 902. In implementations, the processor may perform step 902 similarly to step 602 of sample process 600. The processor identifies ECG feature data points corresponding to an RS segment of a QRS complex candidate at step 904. In implementations, the processor may perform step 904 similarly to step 604 of sample process 600. More specifically, with step 904, the certain ECG feature is an RS segment of an ECG feature identified as a QRS complex candidate (e.g., an ECG feature identified as likely, such as within a certain predetermined amount of certainty, belonging to a QRS complex of a cardiac cycle). The processor determines a minimum ECG morphological region based on the ECG feature data points at step 906. In implementations, the processor may perform step 906 similarly to step 606 of sample process 600. In examples, the minimum ECG morphological region is a minimum convex ECG morphological region, as described above with respect to step 606, although other methods may be used to determine the minimum ECG morphological region in other examples. The processor identifies one or more measurements corresponding to the RS segment using the minimum ECG morphological region at step 908. In implementations, the processor may perform step 908 similarly to step 608 of sample process 600. For instance, the processor may determine the area of the minimum ECG morphological region, such as the area of the minimum convex ECG morphological region. As an illustration, FIG. 13 shows an example of a QRS complex candidate 1000 where a minimum ECG morphological region 1002 has been determined for the RS segment of the QRS complex candidate 1000. The processor may then determine an area of the minimum ECG morphological region 1002 for the RS segment of the QRS complex candidate 1000.

Finally, the processor determines whether the ECG feature data points correspond to a PVC based on the one or more measurements at step 910. For instance, normal QRS complexes may tend to have a smaller minimum ECG morphological region area due to generally being taller but narrower than electrical activity associated with PVCs, which may tend to be shallower but much wider than normal QRS complexes, particularly in the equivalent of the RS region of the electrical activity. As such, the processor may be able to determine at least partially based on the area of the minimum ECG morphological region of the RS complex for the QRS complex candidate whether the QRS complex candidate corresponds to a normal beat or to a PVC. In implementations, the processor may have previously received a predetermined number of QRS complex candidates for the patient 100 that have already been classified as corresponding to normal cardiac cycles or as corresponding to a PVC. The processor may determine the minimum ECG morphological region for each of the classified QRS complex candidates using the process described above with respect to FIG. 12 and determine the area of each minimum ECG morphological region. As shown in FIG. 14, the processor then may construct a histogram 1100 of the counts for the area of minimum ECG morphological regions for QRS complex candidates classified as normal beats and a histogram 1102 of the counts for the area of minimum ECG morphological regions for QRS complex candidates classified as PVCs. Using the histograms 1100 and 1102, the processor may set a threshold for identifying whether a QRS complex candidate corresponds to a normal cardiac cycle or a PVC for the patient 100, depending on a desired sensitivity and specificity. For example, if a higher specificity is desired, the processor may set the threshold 1104 for the patient 100, which will classify some actual PVCs as normal beats but rarely classify an actual normal beat as a PVC. As another example, if a higher sensitivity is desired, the processor may set the threshold 1106 for the patient 100, which will classify some normal beats as PVCs but classify much more PVCs correctly.

In examples, the processor may set the threshold for the patient 100 according to default settings (e.g., a predetermine sensitivity and specificity). In examples, the processor may set the threshold for the patient 100 according to settings received from a caregiver for the patient 100. For instance, the patient's caregiver may input a desired sensitivity and specificity for the patient 100 (e.g., via a user interface of the wearable cardiac sensing device 102 while the wearable cardiac sensing device 102 is being fitted and/or set up for the patient 100, via a caregiver interface 116, etc.). The processor may then set the threshold for the patient 100 according to the sensitivity and specificity input by the patient's caregiver. In examples, the processor may not classify a QRS complex candidate based solely on the histograms 1100 and 1102 but may use the threshold set based on the histograms 1100 and 1102 as an input in a larger beat classifier (e.g., with a predetermined weight given to each input of the beat classifier). In examples, the histograms 1100 and 1102 may not be constructed on a patient-by-patient basis but may instead be constructed from sample patient data.

In implementations, in addition to identifying a minimum ECG morphological region corresponding to the RS segment of a cardiac cycle, the processor may identify a minimum ECG morphological region corresponding to the QR segment of the same cardiac cycle. As such, the processor may identify a second set of ECG feature data points corresponding to the QR segment of the QRS complex candidate, determine a second minimum ECG morphological region (e.g., a convex region or other type of region, as described above) based on the second set of ECG feature data points, and identify another set of one or more measurements corresponding to the QR segment of the QRS complex candidate using the second minimum ECG morphological region. The processor may perform these processes using similar methods described above with respect to steps 604-608 of sample process 600 and steps 904-908 of sample process 900. Therefore, as part of performing step 910, the processor may determine whether the QRS complex candidate corresponds to a PVC or a normal ventricular contraction based both on the measurement(s) corresponding to the RS segment and the measurement(s) corresponding to the QR segment.

FIGS. 13 and 14 provides an illustration of the foregoing. As shown in FIG. 13, the data points forming a QRS complex candidate 1000 have been split into a data points corresponding to a QR segment and an RS segment. A processor has constructed a minimum ECG morphological region 1002 corresponding to the RS segment and a minimum ECG morphological region 1004 corresponding to the QR segment (e.g., as part of performing steps 904 and 906). The processor may then determine a ratio of the area of the minimum ECG morphological region 1004 corresponding to the QR segment and the area of the minimum ECG morphological region 1002 corresponding to the RS segment and identify whether the QRS complex candidate 1000 corresponds to a normal beat or a PVC based at least partially on the ratio.

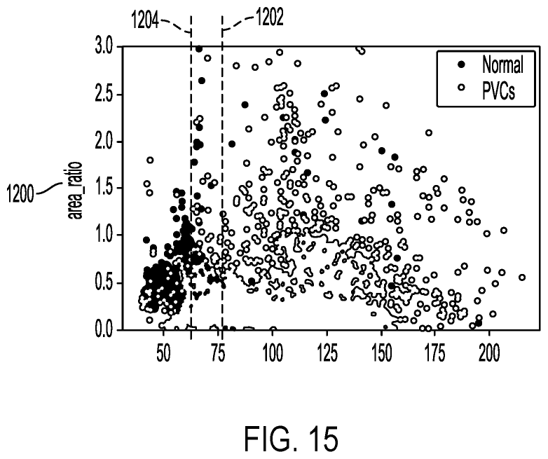

For example, the processor may have previously received a predetermined number of QRS complex candidates for the patient 100 that have already been classified as corresponding to normal cardiac cycles or as corresponding to a PVCs. The processor accordingly may determine the minimum ECG morphological region for the QR segment and the RS segment for each of the classified QRS complex candidates and further generate the ratio of the area of the ECG morphological region for the QR segment to the area of the ECG morphological region for the RS segment of each classified QRS complex candidate. The processor may then plot the ratio of against the QRS duration (e.g., in ms) for each QRS complex candidate. FIG. 15 illustrates an example plot 1200 of the ratio versus QRS duration. Similar to the histograms 1100 and 1102 shown in FIG. 14, the processor may then determine a threshold for classifying QRS complex candidates for the patient 100 based on a desired sensitivity and specificity (e.g., based on default sensitivity and specificity values, based on sensitivity and specificity values received from the patient's caregiver, etc.). For example, if a greater specificity is desired, the processor may set the threshold 1202 for the patient 100. If instead a greater sensitivity is desired, the processor may set the threshold 1204 for the patient. Alternatively, in examples, the plot 1200 may not be constructed on a patient-by-patient basis and instead be constructed from sample patient data. In examples, whether the ratio of the area of the ECG morphological region for the QR segment to the area of the ECG morphological region for the RS segment for a given QRS complex candidate falls on either side of the threshold may be used as an input for a larger beat classifier.

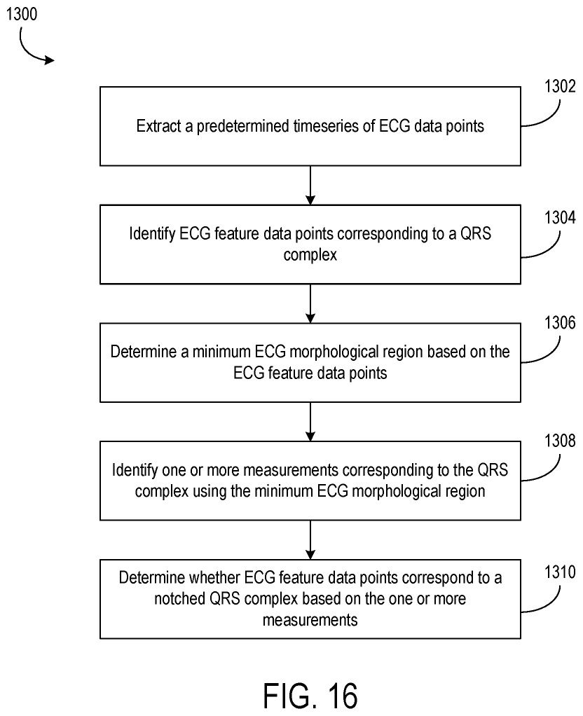

FIG. 16 illustrates a sample process flow for applying improved ECG morphological analysis for identifying notched or fragmented QRS complexes in a patient. Similar to the sample process 600, the sample process 1300 can be implemented by a processor in communication with the wearable cardiac sensing device 102. The processor may be implemented at the wearable cardiac sensing device 102 (e.g., at the cardiac sensing unit 106, portable gateway 110, or garment-based sensing device 118) and/or at the remote server 104.

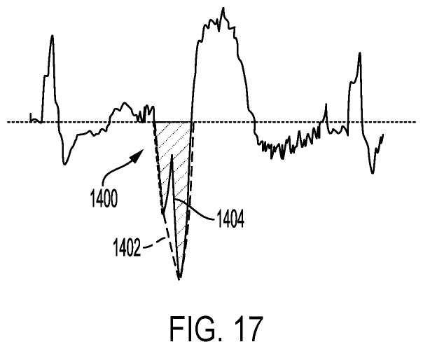

The processor extracts a predetermined timeseries of ECG data points at step 1302. In implementations, the processor may perform step 1302 similarly to step 602 of sample process 600 and step 902 of sample process 900. The processor identifies ECG feature data points corresponding to a QRS complex at step 1304. In implementations, the processor may perform step 1304 similarly to step 604 of sample process 600 and step 904 of sample process 900. More specifically, with step 1304, the certain ECG feature is a QRS complex. The processor determines a minimum ECG morphological region based on the ECG feature data points at step 1306. In implementations, the processor may perform step 1306 similarly to step 606 of sample process 600 and step 906 of sample process 900. In examples, the minimum ECG morphological region is a minimum convex ECG morphological region, as described above with respect to step 606, although other methods may be used to determine the minimum ECG morphological region in other examples. The processor identifies one or more measurements corresponding to the QRS complex using the minimum ECG morphological region at step 1308. In implementations, the processor may perform step 1308 similarly to step 608 of sample process 600 and step 908 of sample process 900. For instance, the processor may determine the area of the minimum ECG morphological region, such as the area of the minimum convex ECG morphological region. As an illustration, FIG. 17 shows an example of a QRS complex 1400 where a minimum ECG morphological region 1402 has been determined for the QRS complex 1400.

Finally, the processor determines whether the ECG feature data points correspond to a notched QRS complex, which may be indicative of previous damage to the patient's heart (e.g., from a myocardial infarction), based on the one or more measurements at step 1310. For instance and referring back to FIG. 17, in addition to determining the minimum ECG morphological region 1402 for the QRS complex 1400, the processor may generate a trace 1404 of the ECG morphological region, where the trace is a polygon constructed by connecting succeeding ECG data points of the QRS complex and an enclosing line segment connecting a first of the QRS complex data points and a last of the QRS complex data points. The processor may determine both an area of the minimum ECG morphological region 1402 and an area of the trace 1404. For normal QRS complexes, the areas of the minimum ECG morphological region and the of the trace may be comparable. However, for notched QRS complexes and as illustrated in FIG. 17 as the QRS complex 1400 is notched, the area of the minimum ECG morphological region may be larger than the area of the trace due to including the area of the notch as part of the minimum ECG morphological region. As such, the processor may determine whether the QRS complex is a normal QRS complex or a notched QRS complex based on whether the area of the minimum ECG morphological region for the QRS complex is within a certain predetermined percentage (e.g., within 95%, within 90%, within 85%, etc.) or within a certain predetermined amount of area of the trace for the QRS complex. In examples, whether the area of the minimum ECG morphological region for the QRS complex is within a certain amount of the area of the trace for the QRS complex may be used as an input in a larger beat classifier.

In implementations, and as discussed above, the processor performing sample processes 600, 900, and/or 1300 may be at the remote server 104 or may be at the wearable cardiac sensing device 102. For example, the wearable cardiac sensing device 102 may perform all of the sample processes 600, 900, and/or 1300 and transmit an indication of an abnormal cardiac event (e.g., an indication of the QRS complex candidate corresponding to a PVC with respect to sample process 900, an indication of the QRS complex being notched with respect to sample process 1300) to the remote server 104. In implementations, the wearable cardiac sensing device 102 may be configured to perform part of sample processes 600, 900, and/or 1300 and transmit data to the remote server 104 to complete the sample processes 600, 900, and/or 1300. As an example, the wearable cardiac sensing device 102 may identify and transmit the ECG feature data points to the remote server 104 for processing.

Returning to the wearable cardiac sensing device 102, in implementations and as discussed above, the wearable cardiac sensing device 102 may include and/or be connected to one or more non-ECG physiological sensors. To illustrate, the wearable cardiac sensing device 102 may include or be connected to a motion sensor (e.g., an accelerometer) configured to sense motions of the patient 100, a cardiovibration sensor configured to sense cardiovibrations of the patient 100, a respiration sensor configured to sense respirations of the patient 100, a thoracic fluid sensor configured to sense thoracic fluid levels in the patient 100, a blood pressure sensor configured to sense a blood pressure of the patient 100, and/or the like. Other examples of non-ECG physiological sensors may include a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

As an illustration, with respect to the wearable cardiac sensing device 102 shown and described above with respect to FIGS. 1-5, the cardiac sensing unit 106 may include and/or be connected to one or more motion sensors. As shown in FIG. 5, the cardiac sensing unit 106 may include, for example, a 3D accelerometer 302 with three axes and a span of ±2 g. Using the 3D accelerometer 302, the cardiac sensing unit 106 may acquire data on the patient's movements and activity level, data on the patient's posture (e.g., angle of posture), data on cardiovibrations of the patient's heart (e.g., vibrations corresponding to the opening and closing of the patient's heart valves), data on the patient's respirations (e.g., data on the patient's respiration rate), and/or the like. In implementations, the cardiac sensing unit 106 may include a different type of motion sensor, such as a 1-axis channel accelerometer, 2-axis channel accelerometer, gyroscope, magnetometer, or ballistocardiography sensor.

As another example, the cardiac sensing unit 106 may include an RF sensor configured to take bio-impedance measurements of the patient's thorax. In implementations, the cardiac sensing unit 106 may then transmit the bio-impedance measurements to the remote server 104, which uses the bio-impedance measurements to determine a thoracic fluid level in the patient 100. In implementations, the cardiac sensing unit 106 may determine a thoracic fluid level in the patient 100 from the bio-impedance measurements and transmit the thoracic fluid level to the remote server 104. Accordingly, as shown in FIG. 5, an example embodiment of the cardiac sensing unit 106 includes at least one RF antenna, such as a transmitting RF antenna 304a and a receiving RF antenna 304b (or a single antenna configured to transmit and receive RF waves, in other implementations), and RF circuitry 306 configured to transmit a low-power signal in an ultra-high frequency band (e.g., 0.1 GHz to 5.0 GHz, 0.5 GHz to 2.1 GHZ) at a predetermined rate (e.g., every 10 ms, every 20 ms, every 30 ms, every 40 ms, every 50 ms, etc.). The cardiac sensing unit 106 may also include other circuits for controlling the RF circuitry 306 (e.g., field-programmable gate array (FPGA) circuits 308). The at least one RF antenna 304a, 304b and RF circuitry 306 receive RF-based biosignals indicative of the thoracic fluid level in the patient 100 in the form of RF waves transmitted through the patient 100 and/or scattered or reflected from the patient 100. For example, the at least one RF antenna 304a, 304b and RF circuitry 306 may detect transmitted, scattered, and/or reflected RF waves for a predetermined amount of time (e.g., about 30 seconds, about 1 minute, about 2 minutes, about 3 minutes, about 5 minutes, about 10 minutes, etc.).

In implementations, the wearable cardiac sensing device 102 (e.g., at the cardiac sensing unit 106 and/or at the portable gateway 110) and/or the remote server 104 are configured to gate when RF measurements are taken and/or discard certain RF measurements based on the patient's state when the RF measurements were taken. For example, the wearable cardiac sensing device 102 and/or the remote server 104 may determine whether the patient 100 showed movement above a predetermined threshold before the wearable cardiac sensing device 102 started the RF measurements process and/or while the RF was taking place. If RF measurements were taken during or immediately after movement above the predetermined threshold, the remote server 104 may discard those RF measurements.

In implementations, the wearable cardiac sensing device 102 shown and described with respect to FIGS. 1-5 may include and/or be connected to other types of non-ECG physiological sensors from the sensors shown in FIG. 5, in addition to the sensors discussed above and/or in the alternative to the sensors discussed above. For example, the wearable cardiac sensing device 102 may include and/or be connected to a bioacoustics sensor, such as a microphone, configured to detect cardiovibrations and/or cardiac acoustics from the patient 100 (e.g., instead of an accelerometer). In implementations, the wearable cardiac sensing device 102 may include and/or be connected to an impedance-based transthoracic sensor configured to sense transthoracic signals indicative of respiration of the patient 100 (e.g., instead of an accelerometer).

In implementations, the wearable cardiac sensing device 102 may include and/or be connected to a blood pressure sensor, such as a blood pressure sensor implemented through an RF sensor (e.g., including the at least one RF antenna 304a, 304b and RF circuitry 306 discussed above) combined with a photoplethysmography (PPG) sensor (e.g., including one or more light emitting diodes (LEDs)). For example, the wearable cardiac sensing device 102 may use the RF sensor to generate information about an aortic waveform (e.g., a waveform of aortic volume over time) and the PPG sensor to generate information about an arterial waveform for arteries a known distance from the aorta. As an illustration, the RF sensor may be provided in the cardiac sensing unit 106 as discussed above, where the cardiac sensing unit 106 and the adhesive patch 108 are located over the patient's sternum. The cardiac sensing unit 106 may transmit RF waves into the patient's thorax and receive reflected and/or scattered RF waves, which the wearable cardiac sensing device 102 uses to generate information about the patient's aortic waveform (e.g., at the cardiac sensing unit 106 and/or the portable gateway 110). The PPG sensor may be provided in the cardiac sensing unit 106 and/or in the adhesive patch 108 and configured to transmit light waves into and receive scattered and/or reflected light waves from surface arteries over the patient's sternum. The wearable cardiac sensing device 102 then uses the received light waves to generate information about the patient's arterial waveform (e.g., at the cardiac sensing unit 106 and/or the portable gateway 110). As another illustration, instead of the cardiac sensing unit 106 and/or adhesive patch 108 including the PPG sensor, the PPG sensor may be provided in another device configured with the PPG sensor, such as a wristband or armband. The device with the PPG sensor may be electronically and/or communicably coupled to the wearable cardiac sensing device 102 and/or to the portable gateway 110.

Once the information about the aortic waveform and the arterial waveform has been generated, the wearable cardiac sensing device 102 may determine the patient's pulse wave velocity by identifying a fiducial point on the aortic waveform (e.g., a point of highest volume or a point of onset in the aorta in a cardiac cycle), identifying a corresponding fiducial point on the arterial waveform (e.g., a point of highest volume or a point of onset in the surface arteries in the same cardiac cycle), determine a time difference between the two fiducial points, and divide the known distance between the aorta and the surface arteries being measured by the time difference between the two fiducial points. The wearable cardiac sensing device 102 may then determine the patient's blood pressure from the pulse wave velocity. Alternatively, the wearable cardiac sensing device 102 may transmit the information about the patient's aortic waveform and the information about the patient's arterial waveform to the remote server 104. The remote server 104 may then perform some or all of this analysis. Additional details on implementations of a blood pressure sensor as described above may be found in U.S. patent application Ser. No. 17/656,480, filed on Mar. 25, 2022, titled “SYSTEM FOR USING RADIOFREQUENCY AND LIGHT TO DETERMINE PULSE WAVE VELOCITY,” which is hereby incorporated by reference. Other examples of sensors configured to sense additional types of signals that may be incorporated into the wearable cardiac sensing device 102 shown and described with respect to FIGS. 1-5 may include temperature sensors, pressure sensors, humidity sensors, and/or the like.

In implementations, the distribution of the externally-applied biosignal sensors of the wearable cardiac sensing device 102 shown and described with respect to FIGS. 1-5 may be different from the examples discussed above. For instance, the ECG pads 202 may be provided on the cardiac sensing unit 106 instead of on the adhesive patch 108. The adhesive patch 108 may accordingly include an aperture or another opening whereby the ECG pads 202 of the cardiac sensing unit 106 may contact the skin of the patient 100. As another illustration, the adhesive patch 108 may include one or more motion sensors, such as the 3D accelerometer 302.

In implementations, the patient 100 may instruct the wearable cardiac sensing device 102 shown and described with respect to FIGS. 1-5 to record symptom events. For example, in embodiments, the patient 100 may provide a symptom input via the portable gateway 110 of the wearable cardiac sensing device 102. As an illustration, the portable gateway 110 may include a visual display and a processor for the portable gateway 110 in communication with the visual display. As such, the portable gateway 110 may be configured to display, on the visual display, a screen that includes a list of potential cardiac-related symptoms to the patient 100. Example symptoms may include lightheadedness, a racing heart, fatigue, fainting, a fall, chest discomfort, a skipped heartbeat, and shortness of breath. The portable gateway 110 further may be configured to receive a selection by the patient 100 of one or more of the displayed potential cardiac-related symptoms. For instance, the visual display may include a touchscreen, where the patient 100 interacts with the touchscreen to select the cardiac-related symptom(s) the patient 100 is experiencing. As another example, the patient 100 may use an input device of the portable gateway 110, such as arrow buttons, a tracking pad, or a keyboard, to select the cardiac-related symptom(s) that the patient 100 is experiencing. The portable gateway 110 thus records the selected cardiac-related symptom(s) and the time the patient 100 submitted the cardiac-related symptom(s).

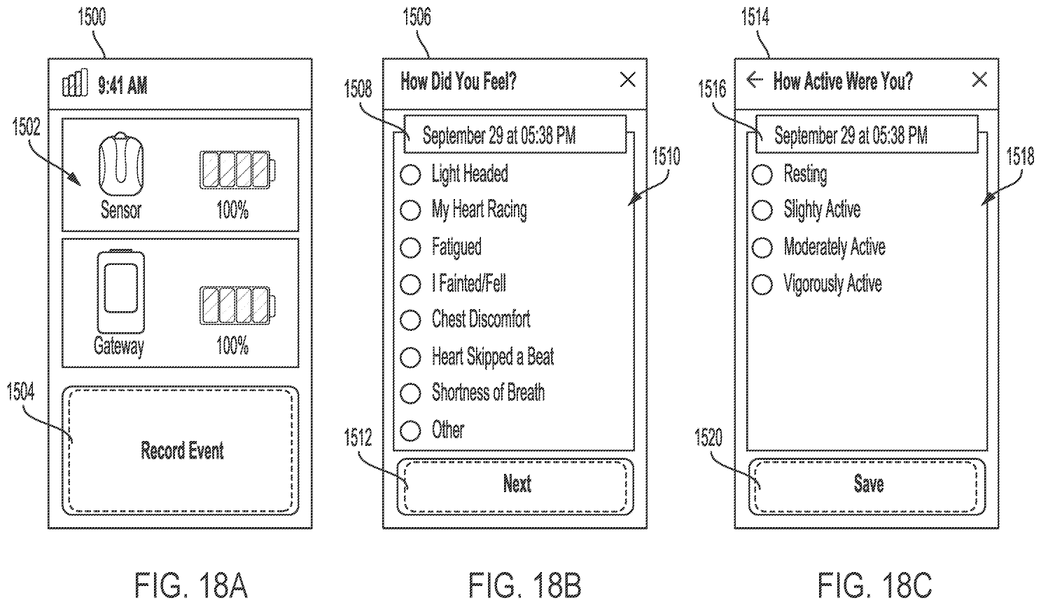

To illustrate, FIGS. 18A-18C show example screens that may be displayed on a visual display of the portable gateway 110 and used by the patient 100 to provide the symptom input. FIG. 18A shows an example home screen 1500 that may be displayed by the portable gateway 110, for instance, as a default screen for the portable gateway 110. The home screen 1500 may include information on the wearable cardiac sensing device 102 such as battery level indicators 1502 (e.g., for the portable gateway 110 and the cardiac sensing unit 106). Additionally, the home screen 1500 includes a “Record Event” button 1504 that the patient 100 can press if the patient 100 is experiencing a suspected cardiac-related symptom.

In response to the patient 100 selecting the “Record Event” button 1504, the portable gateway 110 may display a symptom screen 1506, an example of which is shown in FIG. 18B. The symptom screen may include a subsection 1508 listing the time and date that the patient 100 is reporting the suspected cardiac-related symptom (e.g., the time and date the patient 100 selected the “Record Event” button 1504). In addition, the symptom screen 1506 includes a list 1510 of potential cardiac-related symptoms. As an illustration, in the example of FIG. 18B, the list 1510 of potential cardiac-related symptoms may include Light-Headed,” “My Heart Racing,” “Fatigued,” “I Fainted/Fell,” “Chest Discomfort,” “Heart Skipped a Beat,” “Shortness of Breath,” and “Other” (e.g., in response to the selection of which the patient 100 is shown a screen onto which the patient 100 may type their “other” symptom(s)). The symptom screen 1506 may also include a “Next” button 1512 that the patient 100 can press once the patient 100 has selected the suspected cardiac-related symptom(s) from the list 1510.

In response to the patient 100 selecting the “Next” button 1512, the portable gateway 110 may display an activity screen 1514, an example of which is shown in FIG. 18C. The activity screen 1514 may include a subsection 1516 that again lists the time and date that the patient 100 is reporting the suspected cardiac-related symptom. The activity screen 1514 also includes a list 1518 of types of potential activities that the patient 100 may have been engaging in when the patient 100 started experiencing the recorded symptom(s). As shown in the example of FIG. 18C, the list 1518 of potential types of activity may include “Resting,” “Slightly Active,” “Moderately Active,” and “Vigorously Active.” The activity screen 1514 may also include a “Save” button 1520 that the patient 100 can press once the patient 100 has selected the type of activity the patient 100 was experiencing from the list 1518. Once the patient 100 has selected the “Save” button 1520, the portable gateway 110 may transmit the selected cardiac-related symptom(s) from the list 1510 of potential cardiac-related symptoms, the selected type of activity from the list 1518 of potential types of activity, and the time and date of the symptom input.

In implementations, the example screens described above may be displayed on a user interface disposed on the wearable cardiac sensing device 102, e.g., on a housing of the cardiac sensing unit 106. The patient 100 may thus provide the symptom input via the cardiac sensing unit 106. For example, the front of the cardiac sensing unit 106 (e.g., the face of the cardiac sensing unit 106 facing away from the patient 100 when the cardiac sensing unit 106 is being worn by the patient 100) may be configured to receive a patient input. As an illustration, the front of the cardiac sensing unit 106 may include a button that the patient 100 can tap, or the cardiac sensing unit 106 may sense changes in electrical charge on the front surface by the patient 100 tapping on the front surface. The patient 100 can then provide a symptom input to the cardiac sensing unit 106 via the front face of the cardiac sensing unit 106. An example process for providing a symptom input to the cardiac sensing unit 106 may include (1) the patient 100 remaining still, (2) double tapping the front face of the cardiac sensing unit 106 with the palm of the patient's hand, and (3) waiting for a beeping sound from the cardiac sensing unit 106, which indicates that the symptom input has been recorded. If the beeping sound does not occur, the patient 100 may need to re-tap the cardiac sensing unit 106 to record the symptom input.

Furthermore, in addition to recording the symptom input from the patient 100, the wearable cardiac sensing device 102 is configured to record one or more biosignal segments associated with the symptom input. In implementations, the wearable cardiac sensing device 102 is configured to record the one or more biosignal segments within a predetermined time period before and after the symptom input. For example, the wearable cardiac sensing device 102 may record the one or more biosignal segments 30 seconds, 1 minute, 2 minutes, 3 minutes, 4 minutes, 5 minutes, etc. before and/or after the symptom input. In some cases, the wearable cardiac sensing device 102 may record the one or more biosignal segments for a predetermined time period after the symptom input. In some cases, the wearable cardiac sensing device 102 may record the one or more biosignal segments for a first predetermined time period before the symptom input and a second predetermined time period after the symptom input. The first predetermined time period may be the same as the second predetermined time period, or the first predetermined time period may be different from the second predetermined time period. As an illustration, the wearable cardiac sensing device 102 may record the one or more biosignal segments for 30 seconds before the symptom input and 1 minute after the symptom input. The biosignal(s) for the one or more biosignal segments may include any of the biosignal(s) discussed above, such as ECG signals, cardiovibrational signals, respiration signals, thoracic fluid signals, and/or the like. In implementations, the wearable cardiac sensing device 102 may also record one or more additional signal segments. For example, the wearable cardiac sensing device 102 may record accelerometer signal segments containing activity level and posture information for the patient 100. The wearable cardiac sensing device 102 may be configured to transmit data associated with the symptom event (e.g., including an indication of the symptom event and/or the one or more biosignal segments recorded in association with the symptom event) to the remote server 104.

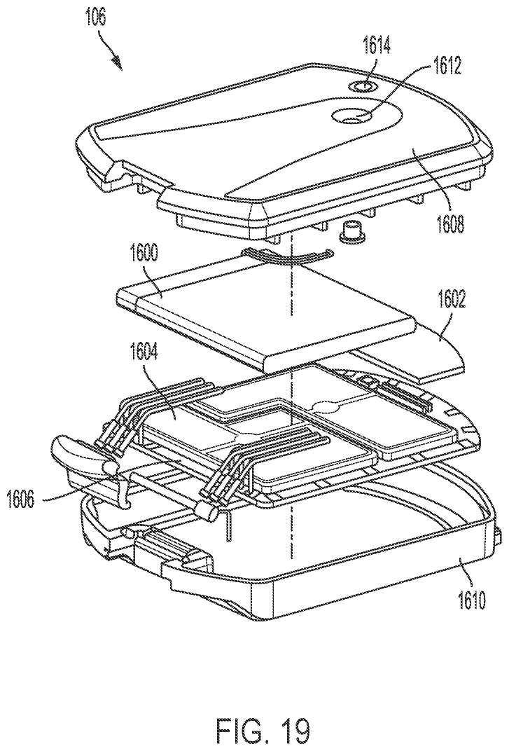

In implementations, the wearable cardiac sensing device 102 of FIGS. 1-5 may also include additional components to those described above. FIG. 19 provides an exploded view of the cardiac sensing unit 106, according to some implementations. The exploded view of FIG. 19 illustrates various components of the cardiac sensing unit 106. For example, the cardiac sensing unit 106 may include a power source, such as a battery 1600. In examples, the battery 1600 may be a rechargeable lithium ion battery configured to supply power for at least one month of continuous or near-continuous recording of the one or more biosignals. The cardiac sensing unit 106 may also include a wireless communications circuit 1602 (e.g., similar to wireless communications circuit 314), a radiofrequency shield 1604 (e.g., a metallic cover, for instance, to prevent interference with RF processing and other digital circuitry), a digital circuit board 1606, and/or the like. The wireless communications circuit 1602 may be a Bluetooth® unit, in some implementations, although in addition to or alternatively to the Bluetooth® unit, other modules facilitating other types of communications (e.g., Wi-Fi, cellular, etc.) may be included in the cardiac sensing unit 106.

In implementations, the cardiac sensing unit 106 may be configured to monitor, record, and transmit data or signals (e.g., the biosignal-based data) to the portable gateway 110 continuously (e.g., via the wireless communications circuit 1602). The cardiac sensing unit 106 monitoring and/or recording additional data may not interrupt the transmission of already acquired data to the portable gateway 110. As such, in implementations, both the monitoring/recording and the transmission processes may occur at the same time or nearly the same time. In implementations, if the cardiac sensing unit 106 does suspend the monitoring and/or recording of additional data while the cardiac sensing unit 106 is transmitting already acquired data to the portable gateway 110, the cardiac sensing unit 106 may then resume monitoring and/or recording of additional data before all of the already-acquired data has been transmitted to the portable gateway 110. To illustrate, the interruption period for the monitoring and/or recording of additional data may be less in comparison to the time it takes the cardiac sensing unit 106 to transmit the already-acquired data (e.g., the interruption period being between about 0% to 80%, about 0% to about 60%, about 0% to about 40%, about 0% to about 20%, about 0% to about 10%, about 0% to about 5%, including values and subranges therebetween, of the monitoring and/or recording period). This moderate interruption period may facilitate the near-continuous monitoring and/or recording of additional data during transmission of already-acquired physiological data. For example, in one scenario, when a measurement time is about two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online tests of previously acquired data. In some implementations, if the previous data have problems, the cardiac sensing unit 106 may notify the patient and/or a remote technician of the problems so that appropriate adjustments can be made.

In implementations, the cardiac sensing unit 106 may be configured to monitor, record, and transmit some data in a continuous or near-continuous manner, as discussed above, while monitoring, recording, and transmitting some other data in a non-continuous manner (e.g., periodically, non-periodically, etc.). As an illustration, the cardiac sensing unit 106 may be configured to record and transmit ECG data from the ECG electrodes 202 continuously or nearly continuously while data from at least one RF antenna and RF circuitry (e.g., the at least one RF antenna 304a, 304b and the RF circuitry 306) is transmitted periodically. For example, RF measurements may be taken only when the patient 100 is in a good position for transmitting and receiving RF waves, such as when the patient 100 is not moving. As such, biosignal-based data including ECG data may be transmitted to the portable gateway 110 (and, via the portable gateway 110, to the remote server 104) continuously or near-continuously as additional ECG data is being recorded, while biosignal-based data including RF data may be transmitted once the RF measuring process is completed. In implementations, monitoring and/or recording of signals by the cardiac sensing unit 106 may be periodic and, in some implementations, may be accomplished as scheduled (e.g., periodically) without delay or latency during the transmission of already acquired data to the portable gateway 110. For example, the cardiac sensing unit 106 may take measurements from the patient 100 and transmit the data generated from the measurements to the portable gateway 110 in a continuous manner as described above.

Additionally, in implementations, the portable gateway 110 may continuously transmit the signals provided by the cardiac sensing unit 106 to the remote server 104. Thus, for example, the portable gateway 110 may transmit the signals from the cardiac sensing unit 106 to the remote server 104 with little or no delay or latency. To this end, in the context of data transmission between the wearable cardiac sensing device 102 and the remote server 104, continuously includes continuous (e.g., without interruption) or near continuous (e.g., within one minute after completion of a measurement and/or an occurrence of an event on the cardiac sensing unit 106). Continuity may also be achieved by repetitive successive bursts of transmission (e.g., high-speed transmission). Similarly, immediate data transmission may include data transmission occurring or done immediately or nearly immediately (e.g., within one minute after the completion of a measurement and/or an occurrence of an event on the cardiac sensing unit 106).

Further, in the context of signal acquisition and transmission by the wearable cardiac sensing device 102, continuously may also include uninterrupted collection of data sensed by the cardiac sensing unit 106 with clinical continuity. In this case, short interruptions in data acquisition of up to one second several times an hour, or longer interruptions of a few minutes several times a day, may be tolerated and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In implementations, the bandwidth of the link between the cardiac sensing unit 106 and the portable gateway 110 may be larger, and in some instances, significantly larger, than the bandwidth of the acquired data to be transmitted via the link (e.g., burst transmissions). Such implementations may ameliorate issues that may arise during link interruptions, periods of reduced/absent reception, etc. In implementations, when transmission is resumed after an interruption, the resumption may be in the form of last-in-first-out (LIFO). In some implementations, the portable gateway 110 additionally may be configured to operate in a store and forward mode, where the data received from the cardiac sensing unit 106 is first stored in an onboard memory of the portable gateway 110 and then forwarded to the remote server 104. In some implementations, the portable gateway 110 may function as a pipeline and pass through data from the cardiac sensing unit 106 immediately to the remote server 104. Further, in implementations, the data from the cardiac sensing unit 106 may be compressed using data compression techniques to reduce memory requirements as well as transmission times and power consumption. In some implementations, the link between the portable gateway 110 and the remote server 104 may similarly be larger, and in some instances, significantly larger, than the bandwidth of the data to be transmitted via the portable gateway 110 and the remote server 104.

Alternatively, the wearable cardiac sensing device 102 including the cardiac sensing unit 106 may not include the portable gateway 110. As such, the cardiac sensing unit 106 may continuously transmit data or signals directly to the remote server 104 using similar processes as those discussed above. Additionally, in implementations, the wearable cardiac sensing device 102 may include a garment-based sensing device 118 that may or may not be in communication with a portable gateway 110. Similar processes may thus also be applied to the garment-based sensing device 118 and/or the portable gateway 110 for the purpose of continuously transmitting data or signals to the remote server 104.

Referring back to FIG. 19, the components of the cardiac sensing unit 106 (e.g., the battery 1600, communications circuit 1602, radiofrequency shield 1604, digital circuit board 1606, and/or the like) may be provided between a front cover 1608 forming an upper surface of the cardiac sensing unit 106 and a back cover 1610 forming a bottom surface of the cardiac sensing unit 106. For instance, the back cover 1610 may be configured to contact the adhesive patch 108, and the front cover 1608 may be configured to face away from the patient 100 such that the front cover 1608 is accessible when the cardiac sensing unit 106 is attached to the adhesive patch 108. In some implementations, an indicator light 1612 and/or a button 1614 may be embedded into the front cover 1608 visible through the upper surface. The indicator light 1612 may provide feedback on the status of the cardiac sensing unit 106 and its components, such as the charging and/or power level of the power source of the cardiac sensing unit 106 (e.g., the battery 1600), the attachment level of the cardiac sensing unit 106 to the adhesive patch 108, the attachment level of the adhesive patch 108 to the surface of the body to which the adhesive patch 108 is attached, etc.

The button 1614 may be configured for the patient 100 and/or a caregiver of the patient 100 to provide feedback to the cardiac sensing unit 106 and/or, via the cardiac sensing unit 106, to the remote server 104. For instance, the button 1614 may allow the patient 100 and/or caregiver to activate or deactivate the cardiac sensing unit 106. In some implementations, the button 1614 may be used to reset the cardiac sensing unit 106, as well as pair the cardiac sensing unit 106 to the portable gateway 110 and initiate communication with the portable gateway 110. In some implementations, the button 1614 may allow a user to set the cardiac sensing unit 106 in an “airplane mode,” for example, by deactivating any wireless communication (e.g., Wi-Fi, Bluetooth®, etc.) with external devices and/or servers, such as the portable gateway 110 and/or the remote server 104.

Referring back to FIG. 5, the example cardiac sensing unit 106 includes additional components to those discussed above with reference to FIG. 5. In implementations, as shown in FIG. 5, the cardiac sensing unit 106 includes one or more external interfaces, either connected to or embedded in the cardiac sensing unit 106. For example, the cardiac sensing unit 106 may include the button or switch 1614 for activating the cardiac sensing unit 106, deactivating the cardiac sensing unit 106, pairing the cardiac sensing unit 106 with the portable gateway 110, receiving a patient input related to a symptom, and/or the like. In implementations, the cardiac sensing unit 106 may also include the indicator light 1612 and a buzzer 324 for providing light and/or audio feedback to a user of the cardiac sensing unit 106 (e.g., in response to the patient 100 activating the button 1614 or tapping the cardiac sensing unit 106 to record that the patient 100 is experiencing symptoms suspected to be related to an arrhythmia).

Further, in some embodiments, the cardiac sensing unit 106 may be connectable to the ECG pads or electrodes 202 coupled to the patient 100 (e.g., connectable to the ECG pads 202 embedded in the adhesive patch 108) and to a charger, such as charger 112, via a charging link 320. For instance, the back cover 1610 of the cardiac sensing unit 106 may implement the charging link 320 via metal contacts configured to connect to the ECG pads 202 when the cardiac sensing unit 106 is attached to the adhesive patch 108 and to a charging power source when the cardiac sensing unit 106 is attached to the charger 112. As discussed above, the ECG circuits 300 may then receive signals from the ECG pads 202 when the cardiac sensing unit 106 is attached to the adhesive patch 108. Alternatively or additionally, in implementations the charging link 320 may be implemented through an inductive circuit configured to charge the cardiac sensing unit 106 via a wireless inductive charging. As shown in FIG. 5, the charging link 320 may be coupled to a power management circuit 322 (e.g., when the cardiac sensing unit 106 is attached to the charger 112, when the cardiac sensing unit 106 is placed in proximity to an inductive charging pad), where the power management circuit 322 is configured to charge an onboard power source such as the battery 1600.

In implementations, at least some of the functionality described above as occurring on the cardiac sensing unit 106 may occur on the portable gateway 110. As an example, at least some of the components or processes described above as being located at and/or performed by the cardiac sensing unit 106 may be located at and/or performed by the portable gateway 110. Thus, the controller of the wearable cardiac sensing device 102 may be implemented through a combination of the cardiac sensing unit 106 and the portable gateway 110.

Returning to the wearable cardiac sensing device 102 shown and described with respect to FIGS. 6-8, any of the externally applied sensors described above as being used or incorporated into the wearable cardiac sensing device 102 of FIGS. 1-5 may also be applied to the wearable cardiac sensing device 102 of FIGS. 6-8. For instance, other examples of externally applied sensors 520 may include a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

As an illustration, the one or more cardiovibration sensors 522 can be configured to detect cardiac or pulmonary vibration information. The one or more cardiovibration sensors 522 can transmit information descriptive of the cardiovibrations (and other types of sensed vibrations) to the sensor interface 504 for subsequent analysis. For example, the one or more cardiovibration sensors 522 can detect the patient's heart valve vibration information (e.g., from opening and closing during cardiac cycles). As a further example, the one or more cardiovibration sensors 522 can be configured to detect cardiovibrational signal values including one or more of S1, S2, S3, and S4 cardiovibrational biomarkers. From these cardiovibrational signal values or heart vibration values, certain heart vibration metrics may be calculated (e.g., at the garment-based sensing device 118 and/or at the remote server 104). These heart vibration metrics may include one or more of electromechanical activation time (EMAT), average EMAT, percentage of EMAT (% EMAT), systolic dysfunction index (SDI), or left ventricular systolic time (LVST). The one or more cardiovibration sensors 522 can also be configured to detect heart wall motion, for instance, by placement of the sensor in the region of the apical beat. In implementations, the one or more cardiovibration sensors 522 can include vibrational sensor configured to detect vibrations from the patient's cardiac and pulmonary system and provide an output signal responsive to the detected vibrations of a targeted organ. For example, the one or more cardiovibration sensors 522 may be configured to detect vibrations generated in the trachea or lungs due to the flow of air during breathing. In implementations, additional physiological information can be determined from pulmonary-vibrational signals such as, for example, lung vibration characteristics based on sounds produced within the lungs (e.g., stridor, crackle, etc.). In implementations, the one or more cardiovibration sensors 522 can include a multi-channel accelerometer, for example, a three-channel accelerometer configured to sense movement in each of three orthogonal axes such that patient movement/body position can be detected and correlated to detected cardiovibrations information.

As another illustration, un implementations, the sensor interface 504 may be connected to one or more motion sensors (e.g., one or more accelerometers, gyroscopes, magnetometers, ballistocardiographs, etc.) as part of the externally applied sensors 520. In implementations, the medical device controller 500 may include a motion detector interface, either implemented separately or as part of the sensor interface 504. For instance, as shown in FIG. 8, the medical device controller 500 may include a motion sensor interface 526 operatively coupled to one or more motion detectors 528 configured to generate motion data, for example, indicative of physical activity performed by the patient 100 and/or physiological information internal to the patient 100. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. For instance, the motion data may include accelerometer counts indicative of physical activity performed by the patient 100, accelerometer counts indicative of respiration rate of the patient 100, accelerometer counts indicative of posture information for the patient 100, accelerometer counts indicative of cardiovibrational information for the patient 100, and/or the like.

The motion sensor interface 526 is configured to receive one or more outputs from the motion sensors 528. The motion sensor interface 526 can be further configured to condition the output signals by, for example, converting analog signals to digital signals (if using an analog motion sensor), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In examples, the motion sensor interface 526 can be configured to filter the signals using a high-pass or band-pass filter to isolate the acceleration of the patient due to movement from the component of the acceleration due to gravity. Additionally, the motion sensor interface 526 can configure the outputs from the motion sensor 528 for further processing. For example, the motion sensor interface 526 can be configured to arrange the output of an individual motion sensor 528 as a vector expressing acceleration components of the x axis, the y-axis, and the z-axis of the motion sensor 528. The motion sensor interface 526 can thus be operably coupled to the processor 518 and configured to transfer the output and/or processed motion signals from the motion sensors 528 to the processor 518 for further processing and analysis.

In implementations, the one or more motion sensors 528 can be integrated into one or more components of the garment-based sensing device 118, either within the medical device controller 500 or external to the medical device controller 500 as shown in FIG. 8. For instance, in some implementations, the one or more motion detectors 528 may be located in or near the ECG electrodes 202. In some implementations, the one or more motion detectors 528 may be located elsewhere on the garment-based sensing device 118. For example, a motion detector 528 may be integrated into the medical device controller 500 (e.g., such that the one or more motion detectors 528 would be located within the housing 502 of the medical device controller 500, as shown in FIG. 8). In some implementations, a motion detector 528 may be integrated into another component of the garment-based sensing device 118, such as a therapy electrode 404, the connection pod 408, and/or the like. In some implementations, a motion detector 528 can be integrated into an adhesive ECG sensing and/or therapy electrode patch.

As described above, the sensor interface 504 and the motion sensor interface 526 can be coupled to any one or combination of external sensors to receive patient data indicative of patient parameters. Once data from the sensors has been received by the sensor interface 504 and/or the motion sensor interface 526, the data can be directed by the processor 518 to an appropriate component within the medical device controller 500. For example, ECG signals collected by the ECG sensors 402 may arrive at the sensor interface 504, and the sensor interface 504 may transmit the ECG signals to the processor 518, which, in turn, relays the patient's ECG data to the cardiac event detector 514. The sensor data can also be stored in the data storage 506 and/or transmitted to the remote server 104 via the network interface 508.

Further, referring back to embodiments of the wearable cardiac sensing device 102 including the garment-based sensing device 118, FIG. 8 includes additional components of the medical device controller 500 to those discussed above. In implementations, the user interface 510 may include one or more physical interface devices, such as input devices, output devices, and combination input/output devices, and a software stack configured to drive operation of the devices. These user interface elements may render visual, audio, and/or tactile content. Thus, the user interface 510 may receive inputs and/or provide outputs, thereby enabling a user to interact with the medical device controller 500.

The medical device controller 500 can also include at least one battery 512 configured to provide power to one or more components integrated in the medical device controller 500. The battery 512 can include a rechargeable multi-cell battery pack. In one example implementation, the battery 512 can include three or more cells (e.g., 2200 mA lithium ion cells) that provide electrical power to the other device components within the medical device controller 500. For example, the battery 512 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 500.

Additionally, the garment-based sensing device 118 shown in FIGS. 6-8 may be configured, in various implementations, to provide therapeutic shocks to the patient 100 upon detecting that the patient 100 is experiencing a treatable arrhythmia. The alarm manager 516 can be implemented using hardware or a combination of hardware and software. For instance, in some examples, the alarm manager 516 can be implemented as a software component that is stored within the data storage 506 and executed by the processor 518. In this example, the instructions included in the alarm manager 516 can cause the processor 518 to configure alarm profiles and notify intended recipients using the alarm profiles. In other examples, the alarm manager 516 can be an application-specific integrated circuit (ASIC) that is coupled to the processor 518 and configured to manage alarm profiles and notify intended recipients using alarms specified within the alarm profiles. Thus, examples of the alarm manager 516 are not limited to a particular hardware or software implementation.

The therapy delivery circuit 530 can be coupled to the therapy electrodes 404 configured to provide therapy to the patient 100. For example, the therapy delivery circuit 530 can include, or be operably connected to, circuitry components that are configured to generate and provide an electrical therapeutic shock. The circuitry components can include, for example, resistors, capacitors, relays and/or switches, electrical bridges such as an H-bridge (e.g., including a plurality of insulated gate bipolar transistors or IGBTs), voltage and/or current measuring components, and other similar circuitry components arranged and connected such that the circuitry components work in concert with the therapy delivery circuit 530 and under the control of one or more processors (e.g., processor 518) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

In implementations, the therapy delivery circuit 530 includes a first high-voltage circuit connecting a first pair of the therapy electrodes 404 and a second high-voltage circuit connecting a second pair of the therapy electrodes 404 such that the first biphasic therapeutic pulse is delivered via the first high-voltage circuit and the second biphasic therapeutic pulse is delivered via the second high-voltage circuit. In implementations, the second high-voltage circuit is configured to be electrically isolated from the first high-voltage circuit. In implementations, the therapy delivery circuit 530 includes a capacitor configured to be selectively connected to the first high-voltage circuit and/or the second high-voltage circuit. As such, the first high-voltage circuit may powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 530 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 μF or larger, or four capacitors of approximately 650 μF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 530 can be configured to perform the switching and pulse delivery operations, e.g., under control of the processor 518. As the energy is delivered to the patient 100, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 530 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

In response to the cardiac event detector 514 described above determining that the patient 100 is experiencing a treatable arrhythmia, the processor 518 is configured to deliver a cardioversion/defibrillation shock to the patient 100 via the therapy electrodes 404. In implementations, the alarm manager 516 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 104 or may be implemented as one or more separate systems). For example, when the processor 518 determines using data from the ECG sensing electrodes 202 that the patient 100 is experiencing a treatable arrhythmia, the alarm manager 516 may issue an alarm via the user interface 510 that the patient 100 is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient 100 that the patient 100 can abort the delivery of the defibrillating shock by interacting with the user interface 510. For instance, the patient 100 may be able to press a user response button or user response buttons on the user interface 510, after which the alarm manager 516 will cease issuing an alert and the medical device controller 500 will no longer prepare to deliver the defibrillating shock.

In implementations, the cardiac event detector 514 is configured to detect when the patient 100 is experiencing a cardiac rhythm change (e.g., an episode of ventricular fibrillation (VF), an episode of ventricular tachycardia (VT), a premature ventricular contraction, and/or the like) in response to a cardiac rhythm disruptive shock (e.g., coordinated by the therapy delivery circuit 530) delivered during a baselining session, as discussed above. Depending on the type of cardiac rhythm change, the processor 518 is configured to deliver a cardioversion/defibrillation shock to the patient 100 via the therapy electrodes 404, as discussed above, to restore the patient's normal cardiac rhythm. For example, if the cardiac rhythm change is VF, the processor 518 is configured to deliver a cardioversion/defibrillation shock to the patient 100.

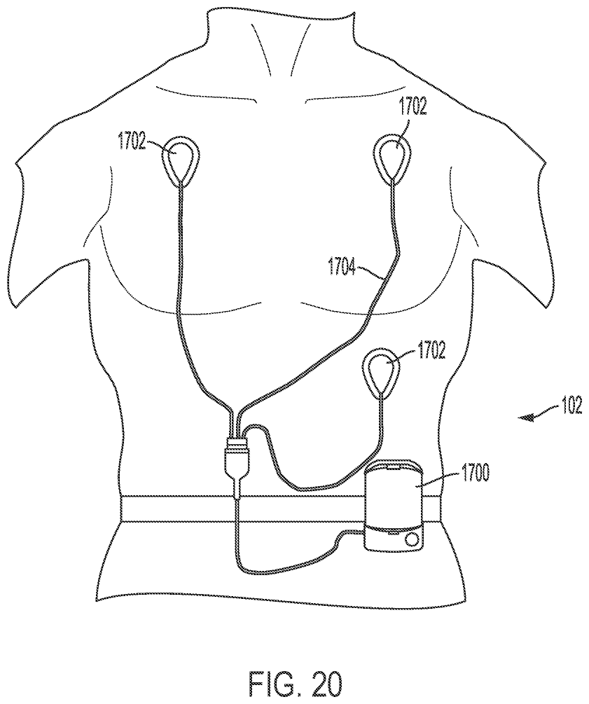

The embodiments of the wearable cardiac sensing device 102 including the cardiac sensing unit 106, adhesive patch 108, and portable gateway 110, and the garment-based sensing device 118, described above are examples of the wearable cardiac sensing device 102. However, other embodiments of a wearable cardiac sensing device 102 may also be used with the systems and methods described herein. Examples of other embodiments of the wearable cardiac sensing device 102, for instance, are described below with respect to FIGS. 20-22.

One alternative implementation of the wearable cardiac sensing device 102 is illustrated in FIG. 20. As shown in FIG. 20, in some implementations, the wearable cardiac sensing device 102 may include a cardiac sensor unit 1700 that further includes individual electrodes 1702 configured to be adhered to the patient's body. Each individual electrode 1702 may be covered with a hydrogel for signal acquisition from the patient. The individual electrodes 1702 may be in wired communication with the sensor unit 1700 via cables 1704, as shown. The sensor unit 1700 may be worn, for example, on a belt of the patient via a belt clip attachment (not shown). In implementations, the sensor unit 1700 may communicate with a portable gateway 110, which transmits signals from the sensor unit 1700 to the remote server 104, similar to implementations discussed above. In implementations, the sensor unit 1700 may communicate directly with the remote server 104, similar to implementations discussed above.

The individual electrodes 1702 may be positioned on the patient in a configuration suited for acquiring ECG signals from the patient. For example, as illustrated in FIG. 20, the wearable cardiac sensing device 102 shown in FIG. 20 may include three electrodes 1702, which two of the electrodes 1702 positioned near either side of the patient's collarbone and the third electrode positioned lower on the patient's thorax. Additionally, in implementations, the wearable cardiac sensing device 102 may include one or more motion sensors in the sensor unit 1700, similar to implementations of the cardiac sensing unit 106 including the adhesive patch 108 discussed above. In implementations, the wearable cardiac sensing device 102 of FIG. 20 may include one or more motion sensors connected to the individual electrodes 1702. For instance, each electrode 1702 may have a connected motion sensor, or a particular electrode 1702 may have a connected motion sensor, such as the electrode 1702 configured to be positioned lower on the patient's thorax in FIG. 20. In implementations, the wearable cardiac sensing device 102 of FIG. 20 may include one or more motion sensors connected to the cables 1704.

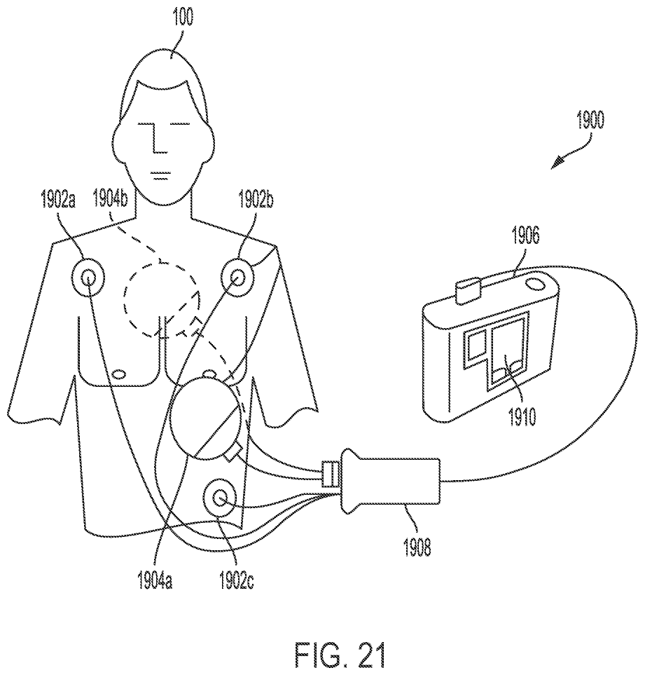

FIG. 21 illustrates another example of a wearable cardiac sensing device 102. More specifically, FIG. 21 shows a hospital wearable defibrillator 1900 that is external, ambulatory, and wearable by the patient 100. Hospital wearable defibrillator 1900 can be configured in some implementations to provide pacing therapy, e.g., to treat bradycardia, tachycardia, and asystole conditions. The hospital wearable defibrillator 1900 can include one or more ECG sensing electrodes 1902a, 1902b, 1902c (e.g., collectively ECG sensing electrodes 1902), therapy electrodes 1904a and 1904b (e.g., collectively therapy electrodes 1904), a medical device controller 1906, and a connection pod 1908. For example, each of these components can be structured and function as similar components of the embodiments of the garment-based sensing device 118 discussed above with reference to FIGS. 6-8. In implementations, the electrodes 1902 and 1904 can include disposable adhesive electrodes. For example, the electrodes 1902 and 1904 can include sensing and therapy components disposed on separate sensing and therapy electrode adhesive patches. In implementations, both sensing and therapy components can be integrated and disposed on a same electrode adhesive patch that is then attached to the patient 100.

As an illustration, the front adhesively attachable therapy electrode 1904a attaches to the front of the patient's torso to deliver pacing or defibrillating therapy. Similarly, the back adhesively attachable therapy electrode 1904b attaches to the back of the patient's torso. In an example scenario, at least three ECG adhesively attachable sensing electrodes 1902 can be attached to at least above the patient's chest near the right arm (e.g., electrode 1902a), above the patient's chest near the left arm (e.g., electrode 1902b), and towards the bottom of the patient's chest (e.g., electrode 1902c) in a manner prescribed by a trained professional. In implementations, the hospital wearable defibrillator 1900 may include additional adhesive therapy electrodes 1904 and/or the patches shown in FIG. 21 may include additional therapy electrodes 1904 on them such that at least two vectors may be formed between the therapy electrodes 1904 of the hospital wearable defibrillator 1900, as described above.

In implementations, the medical device controller 1906 may be configured to function similarly to the medical device controller 406 discussed above with respect to the garment-based sensing device 118. As shown in FIG. 21, the medical device controller 1906 may include a user interface 1910 configured to communicate information with the patient 100. In examples, the patient 100 being monitored by a hospital wearable defibrillator and/or pacing device 1900 may be confined to a hospital bed or room for a significant amount of time (e.g., 75% or more of the patient's stay in the hospital). As a result, a user interface 1910 can be configured to interact with a user other than the patient 100 (e.g., a technician, a clinician or other caregiver) for device-related functions such as initial device baselining (e.g., including performing a baselining therapy session), setting and adjusting patient parameters, and changing the device batteries.

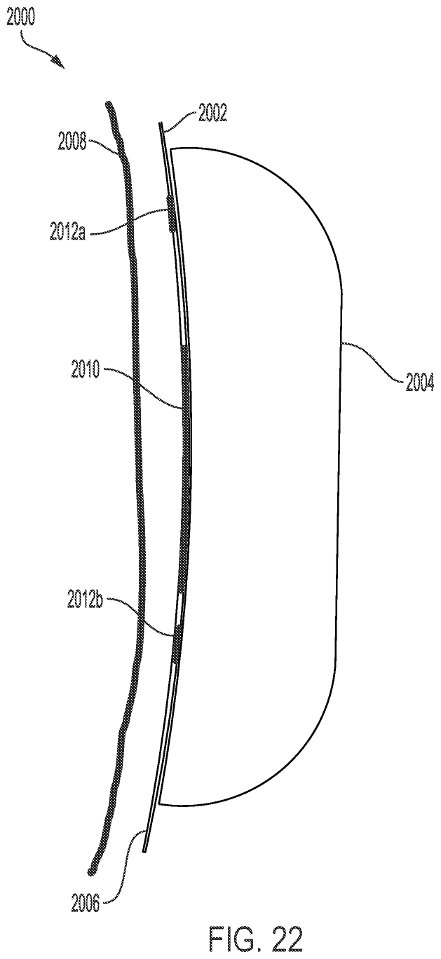

FIG. 22 illustrates another example of a wearable cardiac sensing device 102. As shown in FIG. 22, the wearable cardiac sensing device 102 may be or include an adhesive assembly 2000. The adhesive assembly 2000 includes a contoured pad 2002 and a housing 2004 configured to form a watertight seal with the contoured pad 2002. In implementations, the housing 2004 is configured to house electronic components of the adhesive assembly 2000, such as electronic components similar to components described above with respect to the cardiac sensing unit 106 of FIG. 5 and/or the medical device controller 406 of FIG. 8. The adhesive assembly 2000 includes a conductive adhesive layer 2006 configured to adhere the adhesive assembly 2000 to a skin surface 2008 of the patient 100. The adhesive layer 2006 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 2000 also includes at least one of a therapy electrodes 2010 integrated with the contoured pad 2002. In implementations, the adhesive assembly 2000 may include a therapy electrode 2010 that forms a vector with another therapy electrode disposed on another adhesive assembly 2000 adhered to the patient's body and/or with a separate therapy electrode adhered to the patient's body (e.g., similar to therapy electrodes 404 shown in FIGS. 7 and 8). The adhesive assembly 2000 may also include one or more ECG sensing electrodes 2012 integrated with the contoured pad 2002 (e.g., ECG sensing electrodes 2012a and 2012b). In implementations, the adhesive assembly 2000 may alternatively or additionally be in electronic communication with a separate ECG sensing electrode, such as an adhesive sensing electrode adhered to the patient's body. In examples, as shown in FIG. 22, the therapy electrode(s) 2010 and ECG sensing electrode(s) 2012 may be formed within the contoured pad 2002 such that a skin-contacting surface of each component is coplanar with or protrudes from the patient-contacting face of the contoured pad 2002. Examples of a wearable cardiac sensing device 102 including an adhesive assembly 2000 are described in U.S. patent application Ser. No. 16/585,344, entitled “Adhesively Coupled Wearable Medical Device,” filed on Sep. 27, 2019, which is hereby incorporated by reference in its entirety.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A cardiac event monitoring system for applying improved electrocardiogram (ECG) morphological analysis for determining abnormal cardiac events in a patient, comprising:
a wearable cardiac sensing device configured to be bodily-attached to the patient, the wearable cardiac sensing device comprising
a plurality of physiological sensors comprising one or more ECG electrodes configured to sense electrical signals from a skin surface of the patient;
an ECG digitizing circuit configured to provide digitized ECG signals of the patient based on the sensed electrical signals; and
a non-transitory memory configured to store the digitized ECG signals of the patient; and
a processor in communication with the wearable cardiac sensing device, the processor configured to
extract a predetermined timeseries of ECG data points from the digitized ECG signals;
identify ECG feature data points corresponding to a certain ECG feature from within the digitized ECG signals of the patient;
determine a minimum ECG morphological region based on the ECG feature data points;
identify one or more measurements corresponding to the certain ECG feature using the minimum ECG morphological region; and
determine whether the ECG feature data points correspond to an abnormal cardiac event, based on the one or more measurements corresponding to the certain ECG feature.

2. The cardiac event monitoring system of claim 1, wherein the minimum ECG morphological region comprises a minimum convex ECG morphological region.

3. The cardiac event monitoring system of claim 2, wherein the minimum convex ECG morphological region comprises
a plurality of convex vertices; and
a plurality of line segments interconnecting the plurality of convex vertices and forming an enclosed region;
wherein each of the ECG feature data points is disposed at either a vertex or within the enclosed region.

4. (canceled)

5. (canceled)

6. (canceled)

7. The cardiac event monitoring system of claim 1, wherein the processor is configured to determine the minimum ECG morphological region through morphological closing.

8. The cardiac event monitoring system of claim 1, wherein the wearable cardiac sensing device further comprises a garment configured to be worn around the patient's torso for an extended period of time.

9. (canceled)

10. (canceled)

11. The cardiac event monitoring system of claim 1, wherein the wearable cardiac sensing device further comprises
a removable adhesive patch configured to be adhered to skin of the patient; and
a cardiac sensing unit comprising at least a portion of the plurality of physiological sensors and the processor.

12-34. (canceled)

35. The cardiac event monitoring system of claim 1, wherein the certain ECG feature comprises an RS segment of a QRS complex candidate.

36. The cardiac event monitoring system of claim 35, wherein the abnormal cardiac event comprises a premature ventricular contraction (PVC).

37. (canceled)

38. The cardiac event monitoring system of claim 35, wherein the ECG feature data points comprise first ECG feature data points and the minimum ECG morphological region comprises a first minimum ECG morphological region based on the first ECG feature data points; and
wherein the processor is further configured to identify second ECG feature data points corresponding to a QR segment of the QRS complex candidate from within the digitized ECG signals of the patient.

39. The cardiac event monitoring system of claim 38, wherein the processor is further configured to
determine a second minimum ECG morphological region based on the second ECG feature data points; and
identify one or more measurements corresponding to the QR segment of the QRS complex candidate using the second minimum ECG morphological region.

40. The cardiac event monitoring system of claim 39, wherein the processor is configured to determine whether the QRS complex candidate corresponds to an abnormal cardiac event, based on the one or more measurements corresponding to the RS segment of the QRS complex candidate and further based on the one or more measurements corresponding to the QR segment of the QRS complex candidate.

41. The cardiac event monitoring system of claim 40, wherein the one or more measurements corresponding to the RS segment of the QRS complex candidate comprise an area of the first minimum ECG morphological region, and wherein the one or more measurements corresponding to the QR segment of the QRS complex candidate comprise an area of the second minimum ECG morphological region.

42. The cardiac event monitoring system of claim 41, wherein the processor is configured to determine whether the QRS complex candidate corresponds to an abnormal cardiac event, based on a ratio of the area of the first minimum ECG morphological region and the area of the second minimum ECG morphological region.

43-50. (canceled)

51. The cardiac event monitoring system of claim 1, wherein the certain ECG feature comprises a QRS complex.

52. The cardiac event monitoring system of claim 51, wherein the processor is configured to determine whether the ECG feature data points correspond to an abnormal cardiac event by determining whether the QRS complex comprises a notch, based on the one or more measurements corresponding to the QRS complex.

53. The cardiac event monitoring system of claim 51, wherein the processor is further configured to
generate a trace of the ECG feature data points; and
identify one or more measurements corresponding to the trace of the ECG feature data points.

54. The cardiac event monitoring system of claim 53, wherein the trace comprises a polygon constructed by connecting succeeding ECG feature data points and an enclosing line segment connecting a first of the ECG feature data points and a last of the ECG feature data points.

55. The cardiac event monitoring system of claim 53, wherein the processor is configured to determine whether the ECG feature data points correspond to an abnormal cardiac event, based on the one or more measurements corresponding to the certain ECG feature and further based on the one or more measurements corresponding to the trace of the ECG feature data points.

56. The cardiac event monitoring system of claim 55, wherein the one or more measurements corresponding to the QRS complex comprise an area of the minimum ECG morphological region, and wherein the one or more measurements corresponding to the trace of the ECG feature data points comprise an area of the trace of the ECG feature data points.

57. The cardiac event monitoring system of claim 56, wherein the processor is configured to determine whether the ECG feature data points correspond to an abnormal cardiac event, based on a ratio of the trace of the ECG feature data points and the area of the minimum ECG morphological region.

58-134. (canceled)
